(19) United States
(12) Patent Application Publication
(10) Pub. No.: US 2026/0314640 A1
(43) Pub. Date: Oct. 15, 2026

(54) ELECTROCHEMICAL METHOD FOR MODIFYING REDOX-ACTIVE SENSORY COMPOUNDS AND SYSTEM FOR PERFORMING THE METHOD

(71) Applicant: University of Oregon, Eugene, OR (US)

(72) Inventors: Christopher H. Hendon, Eugene, OR (US); Doran Pennington, Eugene, OR (US)

(73) Assignee: University of Oregon, Eugene, OR (US)

(21) Appl. No.: 19/644,999

(22) Filed: Apr. 10, 2026

Related U.S. Application Data
(60) Provisional application No. 63/787,639, filed on Apr. 11, 2025.

(51) Int. Cl. A23L 5/30; A23F 5/24; C25B 3/05; C25B 3/07; C25B 3/09; C25B 9/17; C25B 9/65; C25B 11/043

(52) U.S. Cl. CPC A23L 5/30; A23F 5/24; C25B 3/05; C25B 3/07; C25B 3/09; C25B 9/17; C25B 9/65; C25B 11/043

(57) ABSTRACT

Disclosed herein are aspects of a method for electrochemically treating a consumable liquid comprising at least one redox-active sensory compound. In some aspects, the method comprises contacting an electrode system with the consumable liquid. In some aspects, the electrode system comprises (i) a working electrode comprising an electroactive material configured to react electrochemically with the at least one redox-active sensory compound; (ii) a counter electrode; and (iii) a power supply. In some aspects, the method further comprises applying a potential between the working electrode and the counter electrode, wherein the potential is predetermined to selectively modify the at least one redox-active sensory compound and thereby change at least one sensory property of the consumable liquid.

### CROSS REFERENCE TO RELATED APPLICATION

This application claims the benefit of and priority to the earlier filing date of U.S. Provisional Application No. 63/787,639, filed Apr. 11, 2025, the entirety of which is herein incorporated by reference in its entirety.

### FIELD

The present disclosure concerns a consumable liquid, systems for electrochemically treating the consumable liquid, and methods of electrochemically treating the consumable liquid.

### BACKGROUND

Various types of beverages, such as coffee and wine, can exhibit inherent variabilities at all stages of preparation, which impacts sensory attributes and perceived quality of the beverages. Coffee preparation, in particular, often faces difficulties in reproducing (i) the brewing process between individual cups, (ii) roast profiles between batches, and (iii) processing methods and growing conditions between crops. As such, coffee quality is challenging to control. Additional difficulties can be associated with preparing other types of beverages, such as wine. Accordingly, there is a need for a method and device that can be used to correct undesirable sensory properties, such as flavor, mouthfeel, texture, viscosity, and aroma, that can stem from both variability in beverage production and undesirable inherent qualities of beverages.

### SUMMARY

Aspects of the present disclosure advantageously provide a system for electrochemically treating a consumable liquid comprising at least one redox-active sensory compound. In certain disclosed aspects, the system comprises a working electrode comprising an electroactive material configured to react electrochemically with the at least one redox-active sensory compound upon exposure of the working electrode to the liquid, a counter electrode, and a power supply configured to apply a potential between the working electrode and the counter electrode. In certain disclosed aspects, the potential is predetermined to selectively modify the at least one redox-active sensory compound and thereby change at least one sensory property of the liquid. In certain disclosed aspects, the working electrode and the counter electrode are positioned to contact the consumable liquid upon exposure of the system to the consumable liquid.

Certain aspects of the present disclosure concern a method for electrochemically treating a consumable liquid comprising at least one redox-active sensory compound. In certain disclosed aspects, the method comprises contacting an electrode system with the consumable liquid, the electrode system comprising (i) a working electrode comprising an electroactive material configured to react electrochemically with the at least one redox-active sensory compound, and (ii) a counter electrode. In certain disclosed aspects, the method comprises applying a potential between the working electrode and the counter electrode. In certain disclosed aspects, the potential is predetermined to selectively modify the at least one redox-active sensory compound and thereby change at least one sensory property of the consumable liquid.

The foregoing and other objects, features, and advantages of the present disclosure will become more apparent from the following detailed description, which proceeds with reference to the accompanying figures.

### BRIEF DESCRIPTION OF THE DRAWINGS

FIGS. 1A-1E show results from treating a coffee sample using a method according to the present disclosure, wherein FIGS. 1A-1C are cyclic voltammetry (CV) plots showing: (i) results obtained at 200 mV/s using boron-doped diamond, glassy carbon, and platinum working electrodes according to aspects of the present disclosure, wherein potential ranges corresponding to caffeine and chlorogenic acids are highlighted, along with protonic features (e.g., hydrogen underpotential deposition (H_{UPD}) and acid redox) (FIG. 1A); (ii) suppression of the protonic features of FIG. 1A with subsequent CV cycling due to deposition of coffee material on the surface of the electrode, wherein total charge can be extracted by subtracting hydrogen evolution reaction (HER) background (see inset) (FIG. 1B); (iii) a relationship between current and mass percentage of total dissolved solids (% TDS) of the brewed coffee (protonic and organic concentration scales with % TDS) (FIG. 1C); a plot illustrating how scanning oxidatively results in mass accumulation on the working electrode, leading to electrode fouling (FIG. 1D); and a mechanism for proton-assisted mass accumulation (FIG. 1E).

FIG. 2 is a plot showing H_{UPD} peaks in undiluted brewed coffee.

FIGS. 3A-3B show plots of peak current over square root of scan rate for a cathodic feature near −0.6 V (FIG. 3A) and an anodic feature near −0.4 V (FIG. 3B).

FIGS. 4A-4B show plots of peak potential separation over scan rate for the cathodic feature near −0.6 V (FIG. 4A) and the anodic feature near −0.4 V (FIG. 4B).

FIG. 5 shows background CV (at a scan rate of 200 mV/s) on water purified using a reverse osmosis system.

FIG. 6 is a plot showing results after subjecting brewed coffee to one CV cycle, with and without sparging, using a method according to aspects of the present disclosure.

FIG. 7 is a plot of CV results (top graph) and associated quartz crystal microbalance (QCM) mass data (bottom graph) obtained from treating a coffee sample using a method according to aspects of the present disclosure.

FIGS. 8A-8C show results from performing evaluations to identify surface adsorbates at negative potentials, wherein: FIG. 8A shows a CV plot (top) and a QCM mass data plot (bottom) summarizing results from performing voltammetry at 200 mV/s sampling potentials more negative than −0.5 V on a coffee sample to determine if accumulated mass on the electrode is maximized rather than liberated back into solution; FIG. 8B is a chromatogram obtained from analyzing the material deposited on the electrode using high-performance liquid chromatography coupled with mass spectrometry (HPLC-MS); and FIG. 8C shows relative binding energies of caffeine and 5-caffeoylquinic acid (5-CQA) on crystalline platinum (Pt) surfaces calculated using density functional theory (DFT).

FIG. 9 shows a calibration curve for quantifying caffeine concentration in a coffee sample after treating the sample using a method according to aspects of the present disclosure, wherein error bars are included from pentaplicate runs and an unknown sample obtained from electrochemical cycling is noted.

FIG. 10 shows a high-resolution mass spectrum corresponding to a chromatographic peak at a retention time of 4.70 minutes; the spectrum was found to be consistent with that of caffeine under the chromatographic conditions used.

FIGS. 11A-11D show results obtained from relating roast profile and beverage strength to a reductive feature at −0.6 V, wherein: FIG. 11A is a plot of measured temperature profiles used to generate six roasted coffee samples that are systematically darker; FIG. 11B is a plot of an electrochemical response of each of the coffees of FIG. 11A after being brewed and diluted to ~1.00±0.02% TDS; FIG. 11C is a plot of total charge passed as a function of % TDS; and FIG. 11D is a plot showing a 3D planar relationship between the total charge and % TDS against the whole bean Agtron value (roast color).

FIG. 12 shows an example of background subtraction to isolate the reduction curve of a CV cycle; the top panel shows a first CV cycle performed on a coffee sample, and the bottom panel shows the reduction sweep of the CV cycle after subtracting a baseline curve that represents background contribution due to onset of hydrogen evolution.

FIG. 13 shows an overlay of a first CV cycle performed on a coffee sample (roast 1a of FIG. 11A) at multiple % TDS values at a scan rate of 200 mV/s.

FIG. 14 shows an overlay of a first CV cycle performed on another coffee sample (roast 1b of FIG. 11A) at multiple % TDS values at a scan rate of 200 mV/s.

FIG. 15 shows an overlay of a first CV cycle performed on another coffee sample (roast 1c of FIG. 11A) at multiple % TDS values at a scan rate of 200 mV/s.

FIG. 16 shows an overlay of a first CV cycle performed on another coffee sample (roast 1d of FIG. 11A) at multiple % TDS values at a scan rate of 200 mV/s.

FIG. 17 shows an overlay of a first CV cycle performed on another coffee sample (roast 1e of FIG. 11A) at multiple % TDS values at a scan rate of 200 mV/s.

FIG. 18 shows an overlay of a first CV cycle performed on another coffee sample (roast 1f of FIG. 11A) at multiple % TDS values at a scan rate of 200 mV/s.

FIG. 19 is a plot of total charge passed as a function of % TDS, with sample points evaluated in triplicate using a single coffee roast sample, wherein the sample was treated according to a method of the present disclosure.

FIG. 20 is a plot of particle size distributions for two preparations of ground coffee according to a method of the present disclosure, wherein the left-most distribution represents ground coffee used for ground bean color assessment, and wherein the right-most distribution represents ground coffee used for preparing brewed coffee.

FIG. 21 is a plot of electrochemical cleaning cycles used for a Pt working electrode used in a device according to aspects of the present disclosure.

FIG. 22 shows a plot comparing CV results for an exemplary coffee sample treated according to a method of the present disclosure, along with background CV scans from (i) water used for brewing and (ii) a solution of sulfuric acid (H₂SO₄) used for electrochemical polishing.

FIG. 23 shows a discriminant analysis (DA) of sensory evaluation data obtained from taste evaluations performed with coffee samples treated using a method according to aspects of the present disclosure.

FIG. 24 shows aspects of variables from DA of sensory evaluation data obtained from taste evaluations performed with coffee samples treated using a method according to aspects of the present disclosure.

FIG. 25 is a plot of two dimensions of DA results obtained from taste evaluations performed with coffee samples treated using a method according to aspects of the present disclosure.

FIG. 26 shows a dendrogram from clustering analysis of sensory evaluation data obtained from taste evaluations performed with coffee samples treated using a method according to aspects of the present disclosure.

FIG. 27 is a chromatogram obtained from analyzing a coffee sample showing different compounds that can be chemically modified using a method according to aspects of the present disclosure.

FIGS. 28A-28D are plots of flavor evolution over time at four different voltages (−2 V in FIG. 28A, −1 V in FIG. 28B, +1 V in FIG. 28C, and +2 V in FIG. 28D, all voltages relative to Ag/AgCl (sat. KCl)) for a dark roast coffee sample treated using a graphite rod working electrode and Pt wire counter electrode according to aspects of the present disclosure.

FIGS. 29A-29D are plots of flavor evolution over time at four different voltages (−2 V in FIG. 29A, −1 V in FIG. 29B, +1 V in FIG. 29C, and +2 V in FIG. 29D, all voltages relative to Ag/AgCl (sat. KCl)) for a medium roast coffee sample treated using a graphite rod working electrode and Pt wire counter electrode according to aspects of the present disclosure.

FIGS. 30A-30D are plots of flavor evolution over time at four different voltages (−2 V in FIG. 30A, −1 V in FIG. 30B, +1 V in FIG. 30C, and +2 V in FIG. 30D, all voltages relative to Ag/AgCl (sat. KCl)) for a light roast coffee sample treated using a graphite rod working electrode and Pt wire counter electrode according to aspects of the present disclosure.

FIGS. 31A-31D are plots of flavor evolution over time at four different voltages (−2 V in FIG. 31A, −1 V in FIG. 31B, +1 V in FIG. 31C, and +2 V in FIG. 31D, all voltages relative to Ag/AgCl (sat. KCl)) for a dark roast coffee sample treated using a Pt mesh working electrode and a Pt wire counter electrode according to aspects of the present disclosure.

### DETAILED DESCRIPTION

### Abbreviations

C: Coulomb (unit of charge)
C_{f}: quartz crystal microbalance (QCM) calibration constant
CQA: caffeoylquinic acid
CVA: Coffee Value Assessment
DA: discriminant analysis
DAHP: 3-deoxy-D-arabino-heptulosonic acid 7-phosphate
DFT: density functional theory
diCQA: dicaffeoylquinic acid
f: frequency
FQA: feruloylquinic acid
fs: femtosecond
HER: hydrogen evolution reaction
H_{UPD}: hydrogen underpotential deposition
M: molar (unit of concentration)
mC: millicoulomb
MD: molecular dynamics
min: minute (unit of time)
MS: mass spectrometry
m/z: mass/charge ratio
nC: nanocoulomb
OER: oxygen evolution reaction
QCM: quartz crystal microbalance
s: second (unit of time)
sat: saturated
SCA: Specialty Coffee Association
SD: standard deviation
% TDS: mass percentage of total dissolved solids

### Overview of Terms

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Unless otherwise indicated, all numbers expressing quantities of components, molecular weights, molarities, voltages, capacities, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing aspects of the disclosure from discussed prior art, the numbers are not approximates unless the word “about” is recited.

Although the operations of some of the aspects of the disclosure are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “introduce,” “flow,” or “provide” to describe the disclosed methods. These terms are high-level abstractions of the actual operations that are performed. The actual operations that correspond to these terms may vary depending on the particular implementation and are readily discernible by one of ordinary skill in the art, particularly with the benefit of the present disclosure.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

Directions and other relative references (e.g., inner, outer, upper, lower, etc.) may be used to facilitate discussion of the drawings and principles herein, but are not intended to be limiting. For example, certain terms may be used such as “inside,” “outside,” “top,” “down,” “interior,” “exterior,” and the like. Such terms are used, where applicable, to provide some clarity of description when dealing with relative relationships, particularly with respect to the illustrated aspects of the disclosure. Such terms are not, however, intended to imply absolute relationships, positions, and/or orientations. For example, with respect to an object, an “upper” part can become a “lower” part simply by turning the object over. Nevertheless, it is still the same part and the object remains the same.

In order to facilitate review of the various aspects of the disclosure, the following explanations of specific terms are provided:

In some aspects, the term “catalyst” generally refers to a substance, usually present in small amounts relative to reactants, which increases the rate of a chemical reaction without itself being consumed or undergoing a chemical change. A catalyst also may enable a reaction to proceed under different conditions (e.g., at a lower temperature) than otherwise possible. In certain aspects, the catalyst can be an associative catalyst or a non-associative catalyst. In certain aspects, the term “associative catalyst” generally refers to a catalyst that forms a complex with reactants during its catalytic cycle, which may lower the activation energy and facilitate the transformation of reactants into products. In certain aspects, the term “non-associative catalyst” generally refers to a catalyst that accelerates the reaction without directly physically associating with the reactants (i.e., without forming a complex with the reactants).

In some aspects, the term “consumable liquid” generally refers to a liquid comprising a food or a beverage. Examples of consumable liquids include coffee, tea, wine, distilled spirits, and soup.

According to the present disclosure, the term “electroanalytical” generally refers to using electrochemistry to measure the concentration of a compound or family of compounds, or measure the chemical environment, using a potential (voltage) at the working electrode. In electroanalytical chemistry, small surface area electrodes (e.g., 1×10⁻⁶ mm² to 20 mm²) are generally favored because the operation is specifically designed to not affect the solution composition (e.g., it is designed merely to measure, it is not designed to change a sensory parameter). The root of this is that while chemical reactions may be reversible, electrochemical reversibility means that the reactions catalyzed with electricity occur both forward and backwards at the same rate. Electroanalytical methods are different from the electrocatalytic methods of the present disclosure because, even if they might result in irreversible oxidation state changes, such irreversible changes do not result in perceptible changes in sensory properties, as the number of molecules affected is below a sensory-detectable concentration, such as on the order of femtomolar concentration or less).

In some aspects, the term “electrocatalytic” or “electrolytic” generally refers to using electrochemistry to drive a chemical reaction in one direction and to permanently change the chemical populations within solution. In certain aspects, the method of the present disclosure uses electrocatalytic chemistry (and not electroanalytical chemistry) to change a structure or composition of a sufficient portion of redox-active sensory compounds present in a sample. In certain aspects, changing the structure or composition comprises reducing, oxidizing, hydrolyzing, decarboxylating (or any combination thereof) the redox-active sensory compound(s). In particular aspects, a “sufficient portion” is one that facilitates a perceptible change in one or more sensory properties of the solution, the perceptible change being a change that can be perceived by a consumer simply by ingesting the solution. In electrocatalytic chemistry, higher surface area electrodes (e.g., 20 mm² and greater) are generally used to perform catalytic reactions.

In some aspects, the term “redox-active sensory compound” generally refers to a compound present in a food or beverage that affects flavor (e.g., flavor profiles described as, for example, floral, fruity, berry, dried fruit, citrus fruit, fermented, green or vegetal, chemical, musty or earthy, papery, roasted, nutty, cocoa, spicy, vanilla, and/or brown sugar, salty, sweet, sour, bitter, umami), mouthfeel (e.g., rough, oily, smooth, mouth-drying, metallic), texture, viscosity, aroma (e.g., aroma profiles described as, for example, floral, fruity, berry, dried fruit, citrus fruit, fermented, green or vegetal, chemical, musty or earthy, papery, roasted, nutty, cocoa, spicy, vanilla, and/or brown sugar), acidity (e.g., dry acidity or sweet acidity), or a combination thereof.

In some aspects, the term “sensory property” generally refers to a parameter associated with sensory perception of a consumable liquid. Examples of sensory properties include flavor (e.g., flavor profiles described as, for example, floral, fruity, berry, dried fruit, citrus fruit, fermented, green or vegetal, chemical, musty or earthy, papery, roasted, nutty, cocoa, spicy, vanilla, and/or brown sugar, salty, sweet, sour, bitter, umami), mouthfeel (e.g., rough, oily, smooth, mouth-drying, metallic), texture, viscosity, aroma (e.g., aroma profiles described as, for example, floral, fruity, berry, dried fruit, citrus fruit, fermented, green or vegetal, chemical, musty or earthy, papery, roasted, nutty, cocoa, spicy, vanilla, and/or brown sugar), acidity (e.g., dry acidity or sweet acidity), or a combination thereof.

### INTRODUCTION

Coffee is just one example of a beverage that exhibits inherent variability in its production and consumption, and this variability can impact sensory attributes and perceived quality for consumers. Despite coffee's popularity, it can be challenging to quantitatively measure chemical properties of coffee related to its gustatory experience.

Beverage concentration and bean color are two independent factors that can dictate coffee sensory perception. Bean color (which correlates to roast level) is readily quantifiable by spectrophotometry. Concentration of solvated coffee can be measured based upon an extracts' refractive index, which can be correlated to an effective concentration through an empirically derived polynomial. The polynomial yields a value generally reported as a mass percentage of total dissolved solids (% TDS) and provides information about strength. From the % TDS, the mass efficiency of extraction can then be estimated (referred to as extraction yield). For example, dissolution of about 20% of dry mass can yield a beverage with about 1.35% TDS, which can generally produce an enjoyable cup of filtered coffee. The refractive index, however, is a function of chemical composition. As such, this approach cannot discern differences between light and dark coffees with the same refractive index, let alone higher-fidelity chemical differences that can result from using modified brewing parameters. Thus, there is a technical need for a quantitative method that reports chemical-specific information beyond % TDS as well as a method and device that can be used to modify sensory parameters of beverages like coffee.

In some instances, chromatography and mass spectrometry can provide qualitative and quantitative information about coffee's chemical composition; however, it can be challenging to identify which compounds correlate to certain sensory experiences. In addition, these methods can suffer from slow run times, high costs, and yield limited predictive insights.

In some instances, electroanalytical methods can be used to probe concentrations of common families of molecules in solution. While electroanalytical methods can measure the solvated concentration of caffeine, chlorogenic acids, and other molecules, it omits the impact of beverage strength, precluding the development of a technique which relates preparation variables (e.g. mass of coffee, mass of water, grind setting, water temperature and pressure, contact time, roast color etc.) to the resultant chemical composition of the liquid.

Accordingly, and in one potential advantage of the present disclosure, changes in the protonic electrochemical response (e.g., hydrogen underpotential deposition (H_{UPD}) and acid redox) in unbuffered aqueous cyclic voltammetry of liquid coffee can be used to quantitatively assess coffee quality and provide more insights than the refractive index.

As described in more detail below, cyclic voltammetry (CV) scans in the reductive region of the water window of brewed coffee produce a response aligned with the deposition of protons on the platinum (Pt) surface before forming hydrogen (H₂). Since Hupp is a function of the number of available surface sites on the Pt, and the concentration of protons (H⁺) in solution, these electrochemical features serve as a useful and sensitive measure of composition in coffee samples. The Pt surface also accumulates coffee material, leading to a reduction in charge passed with subsequent cycling, and at least some fraction of this mass is associated with caffeine and other molecules in coffee, but not the brewing water itself. Density functional theory (DFT) prediction further instructs that this adsorption on the electrode generally has some relationship to molecular composition, and hence different coffees may suppress the reductive feature to different extents. As described in more detail herein, coffees exhibit a linear relationship between beverage strength and total charge passed in the H_{UPD} region (for protonic features that precede hydrogen evolution). The same features can be suppressed with subsequent cycling. The rate of charge suppression with change in % TDS is a function of roasting level. As such, the electrochemistry of coffee is sensitive to the chemical composition of the coffee, which offers a reliable approach to measuring coffee composition and beverage strength (which can, in some aspects, be measured simultaneously).

Furthermore, there is a technical need to correct undesirable flavors stemming from both variability and negative qualities inherent to a coffee once the coffee has been brewed and/or to improve/increase desired flavors or other sensory parameters (e.g., mouthfeel). Accordingly, and in another potential advantage of the present disclosure, electrocatalysis can be applied to a beverage (e.g., brewed coffee) to bring about desirable sensory outcomes within the beverage, with the potential to correct for the variability of beverage preparation and improve the sensory characteristics of the beverage.

### System

In some aspects, a system is provided for electrochemically treating a consumable liquid comprising at least one redox-active sensory compound. In some aspects, the system comprises (i) a working electrode comprising a material configured to react electrochemically with the at least one redox-active sensory compound upon exposure of the working electrode to the liquid; (ii) a counter electrode; and (iii) a power supply configured to apply a potential between the working electrode and the counter electrode, wherein the potential is predetermined to selectively modify the at least one redox-active sensory compound and thereby change at least one sensory property of the liquid. In aspects of the disclosed system, the working electrode and the counter electrode are positioned to contact the consumable liquid upon exposure of the system to the consumable liquid. The power supply may be capable of being connected to/disconnected from the device, or it may be integrated with the device.

In some aspects, the consumable liquid comprises brewed coffee, cold brew coffee, pre-brewed coffee (e.g., a draft latte), espresso or an espresso-based beverage (e.g., an americano or a cappuccino); a different beverage (e.g., wine or other spirits); or a liquid-based food item (e.g., soup). In some aspects, the system can be configured for use in a consumer's home or in a commercial setting and can be used to sense and/or alter a sensory profile of the liquid. For example, in some aspects, the system can be implemented in a coffee machine to correct undesirable flavors in brewed coffee.

In some aspects, the redox-active sensory compound comprises a compound that imparts flavor, mouthfeel, texture, viscosity, aroma, or a combination thereof, to the consumable liquid. In some aspects, the redox-active sensory compound comprises an organic compound, such as a phenol compound, a sugar adduct, a 3-deoxy-D-arabino-heptulosonic acid 7-phosphate (DAHP)-condensate, an alkaloid, an organic acid, or a combination thereof. In some aspects, the redox-active sensory compound is caffeine, a chlorogenic acid, caffeic acid, quinic acid, a decomposition product thereof, or a combination thereof. Some examples of chlorogenic acids that can be modified by the methods disclosed herein include 3-feruloylquinic acid (3-FQA), 4-feruloylquinic acid (4-FQA), 3-caffeoylquinic acid (3-CQA), 4-caffeoylquinic acid (4-CQA), 5-caffeoylquinic acid (5-CQA), 3,4-dicaffeoylquinic acid (3,4-diCQA), 3,5-dicaffeoylquinic acid (3,5-diCQA), and 4,5-dicaffeoylquinic acid (4,5-diCQA).

In some aspects, the working electrode comprises an electroactive material. In some aspects, the electroactive material comprises a carbon material (e.g., glassy carbon, graphite, porous vitreous carbon, diamond, boron-doped diamond, and the like), a metal material (e.g., Pt, Au, Pd, Ag, Ti, stainless steel, metal nanoparticles, and the like), or a combination thereof. In aspects comprising a metal nanoparticle material, the nanoparticle material can comprise nanodots or other metal nanoparticles and they may be deposited on or impregnated within the working electrode. In some aspects, graphite is used as an electroactive material for the working electrode as it can exhibit a greater current response towards substances responsible for “roasty” flavor profiles (e.g., having the flavor or aroma of something, such as malt, that has been roasted, which may include notes of caramel, nuts, and dark chocolate) and potentially bitter-tasting molecules. In some other aspects, platinum (e.g., in wire or mesh form) can be used as the electroactive material for the working electrode as it can enhance perceived fruity flavors and sweetness.

In some aspects, the working electrode comprises an associative catalyst on which the at least one redox-active sensory compound becomes adsorbed upon exposure of the system to the consumable liquid. In some aspects, Pt is an associative catalyst towards caffeine.

In some aspects, the working electrode comprises a non-associative catalyst with which the at least one redox-active sensory compound reacts upon exposure of the system to the consumable liquid. In some aspects, graphitic carbon is a non-associative catalyst towards caffeine.

In some aspects, the counter electrode comprises stainless steel, Pt, Au, Pd, an iridium oxide (e.g., IrO₂), or a combination thereof.

In some aspects, the working electrode and the counter electrode have the same electroactive material composition.

In some aspects, the working electrode and the counter electrode comprise different electroactive material compositions. In some such aspects, the counter electrode can comprise a material that is substantially inert towards the at least one redox-active sensory compound. In some aspects, the counter electrode is a water-splitting electrode, such as a Pt electrode. In certain aspects, the water-splitting electrode greatly favors water electrolysis rather than reacting with the redox-active sensory compound. In this manner, the counter electrode does not affect flavor or other sensory properties of the consumable liquid.

In some aspects, the counter electrode is selected to carry out one or more different electrochemical reaction(s) with the redox-active sensory compound, wherein the electrochemical reaction(s) that take place at the counter electrode are different than those that take place at the working electrode.

In some aspects, the working electrode and/or the counter electrode can be in the form of a mesh, a wire, a rod, or a planar substrate. In some aspects, the working electrode and the counter electrode are the same size and/or shape.

In some aspects, the working electrode and the counter electrode have different sizes and/or shapes. For example, the working electrode can have a greater length, width, height, diameter, surface area, or a combination thereof, when compared to the counter electrode.

In some aspects, the system comprises a two- or three-electrode configuration. The two-electrode configuration comprises a working and counter electrode as described herein. The three-electrode configuration can comprise a further electrode in addition to the working and counter electrode, such as a reference electrode, a pseudoreference electrode, an isolated reference electrode, or a combination thereof. In such configurations, the potential can be predetermined, measured during operation, and/or maintained during operation of the system by use of the reference electrode. Any suitable reference electrode can be used. In some aspects, the reference electrode is an Ag reference electrode. In some aspects, the reference electrode is an Ag/AgCl reference electrode.

In some aspects, the working electrode and/or the counter electrode are pre-treated before being used to make measurements on the redox-active sensory compound and/or modifying the redox-active sensory compound. An example electrode pretreatment protocol comprises one or more steps of completing a cyclic voltammetry scan from 0 V to −1.1 V in coffee at 200 mV/s for 3 cycles, followed by rinsing the electrodes with water and acetone, followed by a cyclic voltammetry scan from 1.1 V to −1.1 V in 50 mM H₂SO₄ at 300 mV/s for one or more cycles, followed by rinsing the electrodes with water and acetone. In this manner, the electrode(s) can be prepared for use. For example, in some aspects, pre-treating the working electrode and/or the counter electrode can increase a rate of electron transfer between the electrode surface and molecule(s) in solution relative to an untreated electrode, increase the effective surface area of the electrode relative to an untreated electrode, and/or remove adsorbed species and contaminants from the electrode surface.

### Method

In some aspects, a method is provided for electrochemically treating a consumable liquid comprising at least one redox-active sensory compound. In some aspects, the method comprises (i) contacting a system according to the present disclosure with the consumable liquid; and (ii) applying a potential between the working electrode and the counter electrode of the system, wherein the potential is predetermined to selectively modify the at least one redox-active sensory compound and thereby change at least one sensory property of the consumable liquid.

In some aspects, different types of consumable liquids can be treated by different potentials or for different durations. For example, two different types of coffees can be electrochemically treated using different parameters according to aspects of the method disclosed herein.

In some aspects, the potential used in the method is commensurate with a redox potential of the redox-active sensory compound. In some aspects, the potential ranges from −10 V to +10 V, including any of the following discrete values, which can further serve as endpoints: −10.0 V, −9.95 V, −9.9 V, −9.85 V, −9.8 V, −9.75 V, −9.7 V, −9.65 V, −9.6 V, −9.55 V, −9.5 V, −9.45 V, −9.4 V, −9.35 V, −9.3 V, −9.25 V, −9.2 V, −9.15 V, −9.1 V, −9.05 V, −9.0 V, −8.95 V, −8.9 V, −8.85 V, −8.8 V, −8.75 V, −8.7 V, −8.65 V, −8.6 V, −8.55 V, −8.5 V, −8.45 V, −8.4 V, −8.35 V, −8.3 V, −8.25 V, −8.2 V, −8.15 V, −8.1 V, −8.05 V, −8.0 V, −7.95 V, −7.9 V, −7.85 V, −7.8 V, −7.75 V, −7.7 V, −7.65 V, −7.6 V, −7.55 V, −7.5 V, −7.45 V, −7.4 V, −7.35 V, −7.3V, −7.25 V, −7.2V, −7.15 V, −7.1 V, −7.05 V, −7.0 V, −6.95 V, −6.9 V, −6.85 V, −6.8 V, −6.75 V, −6.7 V, −6.65 V, −6.6 V, −6.55 V, −6.5 V, −6.45 V, −6.4 V, −6.35 V, −6.3 V, −6.25 V, −6.2 V, −6.15 V, −6.1 V, −6.05 V, −6.0 V, −5.95 V, −5.9 V, −5.85 V, −5.8 V, −5.75 V, −5.7 V, −5.65 V, −5.6 V, −5.55 V, −5.5 V, −5.45 V, −5.4 V, −5.35 V, −5.3 V, −5.25 V, −5.2 V, −5.15 V, −5.1 V, −5.05 V, −5.0 V, −4.95 V, −4.9 V, −4.85 V, −4.8 V, −4.75 V, −4.7 V, −4.65 V, −4.6 V, −4.55 V, −4.5 V, −4.45 V, −4.4 V, −4.35 V, −4.3 V, −4.25 V, −4.2V, −4.15 V, −4.1 V, −4.05 V, −4.0 V, −3.95 V, −3.9 V, −3.85 V, −3.8 V, −3.75 V, −3.7 V, −3.65 V, −3.6 V, −3.55 V, −3.5 V, −3.45 V, −3.4 V, −3.35 V, −3.3 V, −3.25 V, −3.2 V, −3.15 V, −3.1 V, −3.05 V, −3.0 V, −2.95 V, −2.9 V, −2.85 V, −2.8 V, −2.75 V, −2.7 V, −2.65 V, −2.6 V, −2.55 V, −2.5 V, −2.45 V, −2.4 V, −2.35 V, −2.3 V, −2.25 V, −2.2 V, −2.15 V, −2.1 V, −2.05 V, −2.0 V, −1.95 V, −1.9V, −1.85 V, −1.8 V, −1.75 V, −1.7 V, −1.65 V, −1.6 V, −1.55 V, −1.5 V, −1.45 V, −1.4 V, −1.35V, −1.3V, −1.25 V, −1.2V, −1.15 V, −1.1 V, −1.05 V, −1.0 V, −0.95 V, −0.9 V, −0.85 V, −0.8 V, −0.75 V, −0.7 V, −0.65 V, −0.6 V, −0.55 V, −0.5 V, −0.45 V, −0.4 V, −0.35 V, −0.3 V, −0.25 V, −0.2 V, −0.15 V, −0.1V, −0.05 V, 0 V, +0.05 V, +0.1 V, +0.15 V, +0.2 V, +0.25 V, +0.3 V, +0.35 V, +0.4 V, +0.45 V, +0.5 V, +0.55 V, +0.6 V, +0.65 V, +0.7 V, +0.75 V, +0.8 V, +0.85 V, +0.9 V, +0.95 V, +1.0 V, +1.05 V, +1.1V, +1.15V, +1.2V, +1.25 V, +1.3 V, +1.35 V, +1.4 V, +1.45 V, +1.5 V, +1.55 V, +1.6 V, +1.65 V, +1.7 V, +1.75 V, +1.8 V, +1.85 V, +1.9 V, +1.95 V, +2.0 V, +2.05 V, +2.1 V, +2.15 V, +2.2V, +2.25 V, +2.3 V, +2.35 V, +2.4 V, +2.45 V, +2.5 V, +2.55 V, +2.6 V, +2.65 V, +2.7 V, +2.75 V, +2.8 V, +2.85 V, +2.9 V, +2.95 V, +3.0 V, +3.05 V, +3.1 V, +3.15 V, +3.2 V, +3.25 V, +3.3 V, +3.35 V, +3.4 V, +3.45 V, +3.5 V, +3.55 V, +3.6 V, +3.65 V, +3.7 V, +3.75 V, +3.8 V, +3.85 V, +3.9 V, +3.95 V, +4.0 V, +4.05 V, +4.1 V, +4.15 V, +4.2 V, +4.25 V, +4.3 V, +4.35 V, +4.4 V, +4.45 V, +4.5 V, +4.55 V, +4.6 V, +4.65 V, +4.7 V, +4.75 V, +4.8 V, +4.85 V, +4.9 V, +4.95 V, +5.0 V, +5.05 V, +5.1 V, +5.15 V, +5.2 V, +5.25 V, +5.3 V, +5.35 V, +5.4 V, +5.45 V, +5.5 V, +5.55 V, +5.6 V, +5.65 V, +5.7 V, +5.75 V, +5.8 V, +5.85 V, +5.9 V, +5.95 V, +6.0 V, +6.05 V, +6.1 V, +6.15 V, +6.2 V, +6.25 V, +6.3 V, +6.35 V, +6.4 V, +6.45 V, +6.5 V, +6.55 V, +6.6 V, +6.65 V, +6.7 V, +6.75 V, +6.8 V, +6.85 V, +6.9 V, +6.95 V, +7.0 V, +7.05 V, +7.1 V, +7.15V, +7.2 V, +7.25 V, +7.3 V, +7.35 V, +7.4 V, +7.45 V, +7.5 V, +7.55 V, +7.6 V, +7.65 V, +7.7 V, +7.75 V, +7.8 V, +7.85 V, +7.9 V, +7.95 V, +8.0 V, +8.05 V, +8.1 V, +8.15 V, +8.2 V, +8.25 V, +8.3 V, +8.35 V, +8.4 V, +8.45 V, +8.5 V, +8.55 V, +8.6 V, +8.65 V, +8.7 V, +8.75 V, +8.8 V, +8.85 V, +8.9 V, +8.95 V, +9.0 V, +9.05 V, +9.1 V, +9.15 V, +9.2 V, +9.25 V, +9.3 V, +9.35 V, +9.4 V, +9.45 V, +9.5 V, +9.55 V, +9.6 V, +9.65 V, +9.7 V, +9.75 V, +9.8 V, +9.85 V, +9.9 V, +9.95 V, or +10.0 V. In certain aspects, the potential ranges from −2.5 V to +2.5 V. In certain aspects, the potential ranges from +0.35 V to +0.7 V. In certain aspects, the potential is calibrated to an Ag/AgCl reference electrode, regardless of whether the reference electrode is present in the system (e.g., in a three-electrode system) or absent in the system (e.g., in a two-electrode system).

In certain aspects, positive potentials with a carbon working electrode can result in chlorogenic acid oxidation. This oxidation can affect bitterness, astringency, acidity, sweetness and mouthfeel of the consumable liquid. In addition, chlorogenic acids (and other redox-active sensory compounds) can interact with other species in solution. As a result, targeting any one or more redox-active sensory compounds can result in additional sensory changes, such as gaining access to “hidden flavors” that are not present in untreated consumable liquids.

In some aspects, applying a negative potential (e.g., a negative potential ranging from −1.5 V to −2.5 V) can reduce oxidized compounds. This can impart fresh flavors and remove roasted flavors (e.g., by removing sensory compounds that impart burnt or over-roasted flavors). Potentials greater than about-2 V may favor water splitting versus reacting with the one or more redox-active sensory compounds. In certain aspects, the potential can be −1.5 V, −1.2 V, −1 V, −650 mV, −500 mV, −300 mV, +350 mV, +500 mV, +650 mV, +700 mV, +800 mV, +850 mV, +1 V, +1.4 V, +1.5 V, +2 V, +2.5 V, +3 V, or +4 V. In certain aspects, the potential is calibrated to an Ag/AgCl reference electrode, regardless of whether the reference electrode is present in the system (e.g., in a three-electrode system) or absent in the system (e.g., in a two-electrode system).

In some aspects, the consumable liquid is treated with a current ranging from greater than 0 nA/mL to 10 A/mL, including any of the following discrete values, which can also serve as endpoints: 0.05 A/mL, 0.1 A/mL, 0.15 A/mL, 0.2 A/mL, 0.25 A/mL, 0.3 A/mL, 0.35 A/mL, 0.4 A/mL, 0.45 A/mL, 0.5 A/mL, 0.55 A/mL, 0.6 A/mL, 0.65 A/mL, 0.7 A/mL, 0.75 A/mL, 0.8 A/mL, 0.85 A/mL, 0.9 A/mL, 0.95 A/mL, 1.0 A/mL, 1.05 A/mL, 1.1 A/mL, 1.15 A/mL, 1.2 A/mL, 1.25 A/mL, 1.3 A/mL, 1.35 A/mL, 1.4 A/mL, 1.45 A/mL, 1.5 A/mL, 1.55 A/mL, 1.6 A/mL, 1.65 A/mL, 1.7 A/mL, 1.75 A/mL, 1.8 A/mL, 1.85 A/mL, 1.9 A/mL, 1.95 A/mL, 2.0 A/mL, 2.05 A/mL, 2.1 A/mL, 2.15 A/mL, 2.2 A/mL, 2.25 A/mL, 2.3 A/mL, 2.35 A/mL, 2.4 A/mL, 2.45 A/mL, 2.5 A/mL, 2.55 A/mL, 2.6 A/mL, 2.65 A/mL, 2.7 A/mL, 2.75 A/mL, 2.8 A/mL, 2.85 A/mL, 2.9 A/mL, 2.95 A/mL, 3.0 A/mL, 3.05 A/mL, 3.1 A/mL, 3.15 A/mL, 3.2 A/mL, 3.25 A/mL, 3.3 A/mL, 3.35 A/mL, 3.4 A/mL, 3.45 A/mL, 3.5 A/mL, 3.55 A/mL, 3.6 A/mL, 3.65 A/mL, 3.7 A/mL, 3.75 A/mL, 3.8 A/mL, 3.85 A/mL, 3.9 A/mL, 3.95 A/mL, 4.0 A/mL, 4.05 A/mL, 4.1 A/mL, 4.15 A/mL, 4.2 A/mL, 4.25 A/mL, 4.3 A/mL, 4.35 A/mL, 4.4 A/mL, 4.45 A/mL, 4.5 A/mL, 4.55 A/mL, 4.6 A/mL, 4.65 A/mL, 4.7 A/mL, 4.75 A/mL, 4.8 A/mL, 4.85 A/mL, 4.9 A/mL, 4.95 A/mL, 5.0 A/mL, 5.05 A/mL, 5.1 A/mL, 5.15 A/mL, 5.2 A/mL, 5.25 A/mL, 5.3 A/mL, 5.35 A/mL, 5.4 A/mL, 5.45 A/mL, 5.5 A/mL, 5.55 A/mL, 5.6 A/mL, 5.65 A/mL, 5.7 A/mL, 5.75 A/mL, 5.8 A/mL, 5.85 A/mL, 5.9 A/mL, 5.95 A/mL, 6.0 A/mL, 6.05 A/mL, 6.1 A/mL, 6.15 A/mL, 6.2 A/mL, 6.25 A/mL, 6.3 A/mL, 6.35 A/mL, 6.4 A/mL, 6.45 A/mL, 6.5 A/mL, 6.55 A/mL, 6.6 A/mL, 6.65 A/mL, 6.7 A/mL, 6.75 A/mL, 6.8 A/mL, 6.85 A/mL, 6.9 A/mL, 6.95 A/mL, 7.0 A/mL, 7.05 A/mL, 7.1 A/mL, 7.15 A/mL, 7.2 A/mL, 7.25 A/mL, 7.3 A/mL, 7.35 A/mL, 7.4 A/mL, 7.45 A/mL, 7.5 A/mL, 7.55 A/mL, 7.6 A/mL, 7.65 A/mL, 7.7 A/mL, 7.75 A/mL, 7.8 A/mL, 7.85 A/mL, 7.9 A/mL, 7.95 A/mL, 8.0 A/mL, 8.05 A/mL, 8.1 A/mL, 8.15 A/mL, 8.2 A/mL, 8.25 A/mL, 8.3 A/mL, 8.35 A/mL, 8.4 A/mL, 8.45 A/mL, 8.5 A/mL, 8.55 A/mL, 8.6 A/mL, 8.65 A/mL, 8.7 A/mL, 8.75 A/mL, 8.8 A/mL, 8.85 A/mL, 8.9 A/mL, 8.95 A/mL, 9.0 A/mL, 9.05 A/mL, 9.1 A/mL, 9.15 A/mL, 9.2 A/mL, 9.25 A/mL, 9.3 A/mL, 9.35 A/mL, 9.4 A/mL, 9.45 A/mL, 9.5 A/mL, 9.55 A/mL, 9.6 A/mL, 9.65 A/mL, 9.7 A/mL, 9.75 A/mL, 9.8 A/mL, 9.85 A/mL, 9.9 A/mL, 9.95 A/mL, or 10 A/mL. In some aspects, a certain volume of the consumable liquid is treated with a current ranging from greater than 0 mA to 50 mA.

In some aspects, the potential is applied for a time period suitable to pass a charge that can modify the at least one redox-active sensory compound and thereby change at least one sensory property of the consumable liquid. In some aspects, applying the potential comprises passing, into the consumable liquid, a charge ranging from 1 nanocoulomb (nC) per milliliter (mL) of the consumable liquid to 1 C/mL. In some aspects, applying the potential comprises passing, into the consumable liquid, a charge ranging from 100 nC per gram TDS (determined by refractive index) to 4 C/g TDS. In some aspects, a charge ranging from 10 nC to 100 nC per sample (e.g., 40 grams of prepared espresso, or approximately 40 mL) can be suitable to provide a modification of redox-active sensory compounds at a level where a human (e.g., a consumer) can begin to perceive differences in a consumable liquid, particularly for coffee. In some aspects, charges ranging from 1 millicoulomb (mC) to 1 Coulomb (C) or more can have a more substantial effect on flavor than the charge ranging from 10 nC to 100 nC.

In some aspects, the potential is applied for a time period ranging from greater than 0 seconds to 180 seconds, including any of the following discrete values or endpoints: 10 seconds, 20 seconds, 30 seconds, 40 seconds, 50 seconds, 60 seconds, 70 seconds, 80 seconds, 90 seconds, 100 seconds, 110 seconds, 120 seconds, 130 seconds, 140 seconds, 150 seconds, 160 seconds, 170 seconds, or 180 seconds. In certain aspects, the time ranges from 30 seconds to 120 seconds. In certain aspects, the reaction of the at least one redox-active sensory compound occurs instantaneously or substantially instantaneously (e.g., within 1 millisecond (ms) to 100 ms) upon application of the selected potential. It will be appreciated that charge passed is a function of time and surface area. For instance, if there is a sufficiently large (approaching infinite) surface area available relative to volume of the redox-active sensory compound, the electrochemical modification of the redox-active sensory compound may occur substantially instantaneously.

In certain aspects, the time period is a function of concentration of sensory compounds in the consumable liquid. For example, an espresso drink can be treated for a longer duration than an americano drink, as coffee content in the espresso typically is more highly concentrated. One example of an electrochemical treatment includes applying a potential for 60 seconds to a dilute cup of Robusta coffee. In certain such aspects, applying a potential for longer than two or three minutes can cause unsatisfactory levels of electrochemical decay in the consumable liquid.

In some aspects, modifying the at least one redox-active sensory compound comprises electrocatalytically inducing a structural change or a compositional change in the at least one redox-active sensory compound. In certain aspects, electrocatalytically inducing the structural change or the compositional change comprises reducing, oxidizing, hydrolyzing, decarboxylating (or any combination thereof) the at least one redox-active sensory compound.

In some aspects, the method includes one or more of the following steps: (i) measuring dissolved products in a container comprising a consumable liquid, with some aspects including cold brew coffee, pre-brewed coffee, or draft espresso drinks (e.g., latte), either at a brewing factory or at home in a coffee machine; (ii) evaluating the measurement to inform the electrochemical treatment of the consumable liquid. In some aspects, this involves attaining a target electrochemical profile or other analytical profile, applying a set potential, and monitoring the current to show how many reactions are occurring. More charge can be applied if a desired electrochemical profile is not attained after a first treatment.

In some aspects, measuring dissolved products in the consumable liquid can be performed using CV or other electrochemical methods. For example, and as described herein, CV can be used to directly measure ensemble strength of a coffee beverage and, separately, how dark the coffee has been roasted. The CV results can be used to inform the electrochemical treatment of the consumable liquid to attain one or more different sensory properties through electrochemical modification of at least one redox-active sensory compound within the consumable liquid.

### Overview of Several Aspects

A system for electrochemically treating a consumable liquid comprising at least one redox-active sensory compound, the system comprising: a working electrode comprising an electroactive material configured to react electrochemically with the at least one redox-active sensory compound upon exposure of the working electrode to the liquid; a counter electrode; and a power supply configured to apply a potential between the working electrode and the counter electrode, wherein the potential is predetermined to selectively modify the at least one redox-active sensory compound and thereby change at least one sensory property of the liquid; wherein the working electrode and the counter electrode are positioned to contact the consumable liquid upon exposure of the system to the consumable liquid.

In any or all aspects, the electroactive material comprises a carbon material, Pt, Au, Pd, Ag, IrO₂, stainless steel, or a combination thereof.

In any or all aspects, the electroactive material comprises glassy carbon, graphite, porous vitreous carbon, diamond, or boron-doped diamond.

In any or all aspects, the working electrode comprises an associative catalyst on which the at least one redox-active sensory compound becomes adsorbed upon exposure of the system to the consumable liquid.

In any or all aspects, the working electrode comprises a non-associative catalyst with which the at least one redox-active sensory compound reacts upon exposure of the system to the consumable liquid.

In any or all aspects, the working electrode comprises a plurality of nanoparticles.

In any or all aspects, the at least one redox-active sensory compound is a compound that imparts flavor, mouthfeel, texture, viscosity, aroma, or a combination thereof, to the consumable liquid.

In any or all aspects, the system further comprises a reference electrode, and wherein the potential is predetermined relative to the reference electrode.

In any or all aspects, the reference electrode is an Ag/AgCl reference electrode.

In any or all aspects, the counter electrode comprises a different electroactive material than the working electrode.

In any or all aspects, the electroactive material of the working electrode is a carbon material, Pt, Au, Pd, Ag, stainless steel, or a combination thereof, and wherein the electroactive material of the counter electrode is stainless steel, Pt, Au, Pd, IrO₂, or a combination thereof.

In any or all aspects, the counter electrode is substantially inert towards the at least one redox-active sensory compound.

In any or all aspects, the counter electrode has a different size or surface area than the working electrode.

Also disclosed is a method for electrochemically treating a consumable liquid comprising at least one redox-active sensory compound, the method comprising: contacting an electrode system with the consumable liquid, the electrode system comprising (i) a working electrode comprising an electroactive material configured to react electrochemically with the at least one redox-active sensory compound, and (ii) a counter electrode; and applying a potential between the working electrode and the counter electrode, wherein the potential is predetermined to selectively modify the at least one redox-active sensory compound and thereby change at least one sensory property of the consumable liquid.

In any or all aspects, the potential is commensurate with a redox potential of the redox-active sensory compound.

In any or all aspects, the potential ranges from +10 V to −10 V relative to an Ag/AgCl reference electrode.

In any or all aspects, applying the potential comprises passing a charge ranging from 1 nC/mL to 1 C/mL into the consumable liquid.

In any or all aspects, the power supply is configured to apply a current ranging from greater than 0 nA/mL to 10 A/mL between the working electrode and the counter electrode.

In any or all aspects, modifying the at least one redox-active sensory compound comprises electrochemically inducing a structural change or a compositional change in the at least one redox-active sensory compound.

In any or all aspects, electrochemically inducing the structural change or the compositional change comprises reducing, oxidizing, hydrolyzing, or decarboxylating the at least one redox-active sensory compound, or any combination thereof.

In any or all aspects, the at least one redox-active sensory compound is an organic compound.

In any or all aspects, the at least one redox-active sensory compound is a phenol compound, a sugar adduct, a 3-deoxy-D-arabino-heptulosonic acid 7-phosphate (DAHP)-condensate, an alkaloid, an organic acid, or a combination thereof.

In any or all aspects, the at least one redox-active sensory compound is caffeine, a chlorogenic acid, or a combination thereof.

In any or all aspects, the chlorogenic acid is 3-feruloylquinic acid, 4-feruloylquinic acid, 3-caffeoylquinic acid, 4-caffeoylquinic acid, 5-caffeoylquinic acid, 3,4-dicaffeoylquinic acid, 3,5-dicaffeoylquinic acid, or 4,5-dicaffeoylquinic acid.

Also disclosed is a consumable liquid produced by the method according to any or all of the above aspects.

Also disclosed is a system for electrochemically treating a consumable liquid comprising at least one redox-active sensory compound, the system comprising: a working electrode comprising a first electroactive material configured to react electrochemically with the at least one redox-active sensory compound upon exposure of the working electrode to the liquid; a reference electrode; a counter electrode that comprises a second electroactive material that is different from the first electroactive material; and a power supply configured to apply a potential between the working electrode and the counter electrode, wherein the potential is predetermined relative to the reference electrode to selectively modify the at least one redox-active sensory compound and thereby change at least one sensory property of the consumable liquid; wherein (i) the counter electrode is substantially inert towards the at least one redox-active sensory compound, and (ii) the working electrode, the reference electrode, and the counter electrode are positioned to contact the consumable liquid upon exposure of the system to the consumable liquid.

### Examples

Aspects of the present teachings can be further understood in light of the following non-limiting examples.

### Example 1—Assessment of Voltammetric Features Present in Coffee Extracts

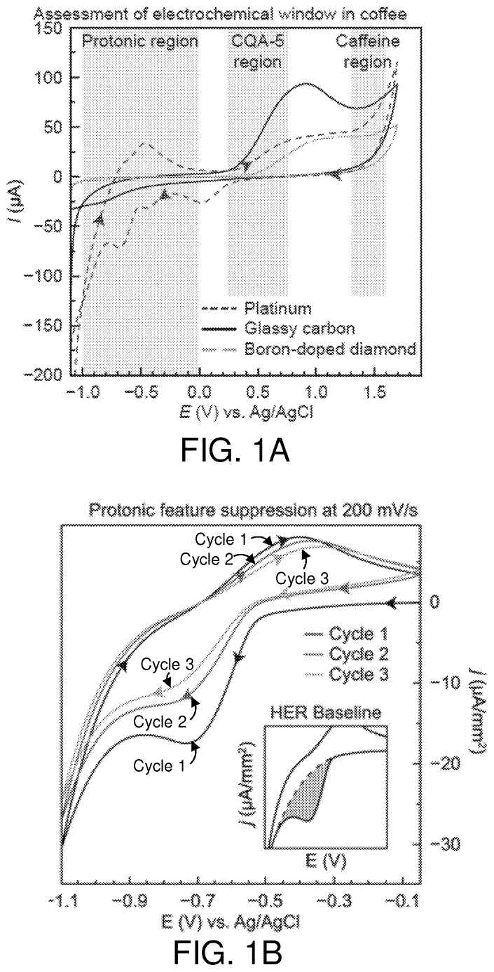

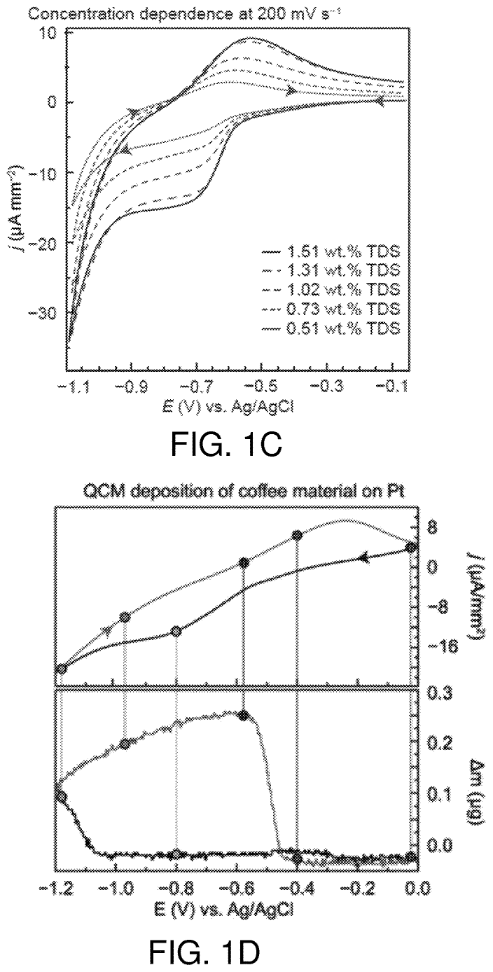

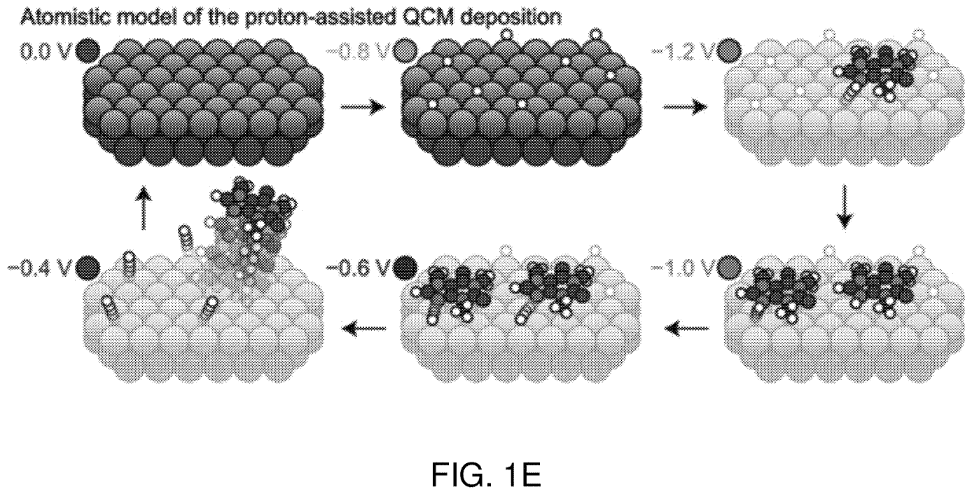

Initially, direct measurements of the concentrations of caffeine (Eₒₓ about 1.4 V vs. Ag/AgCl), chlorogenic acids (Eₒₓ about 0.2 V-0.5 V vs. Ag/AgCl, depending on isomer), and other redox-active species in undiluted samples were conducted to study their relationship to brewing process parameters. Caffeine and chlorogenic acids can form aggregates at concentrations typical of brewed coffee, which terminally impacts their redox activity-they only become electrochemically well-resolved in acidified dilute solutions with added electrolyte (e.g., diluted to below 0.02% TDS). In some instances, boron-doped diamond and glassy carbon electrodes can provide molecule-specific information, but since the primary goal is to measure features of as-consumed coffee, the redox landscape was ascertained by running cyclic voltammetry using boron-doped diamond, glassy carbon, and platinum across a range of potentials afforded to liquid coffee (FIG. 1A).

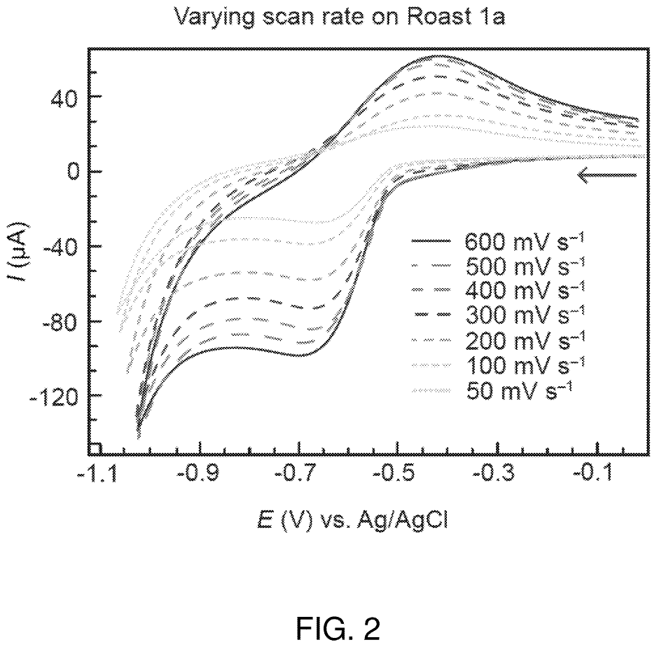

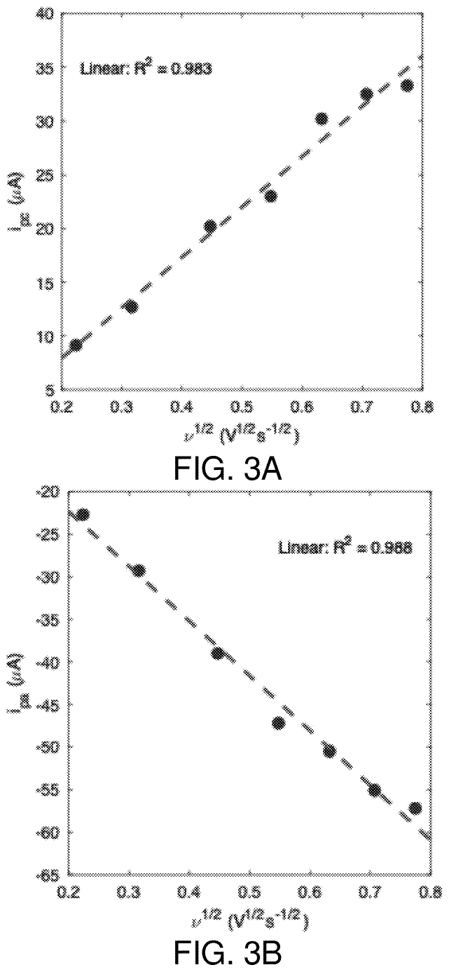

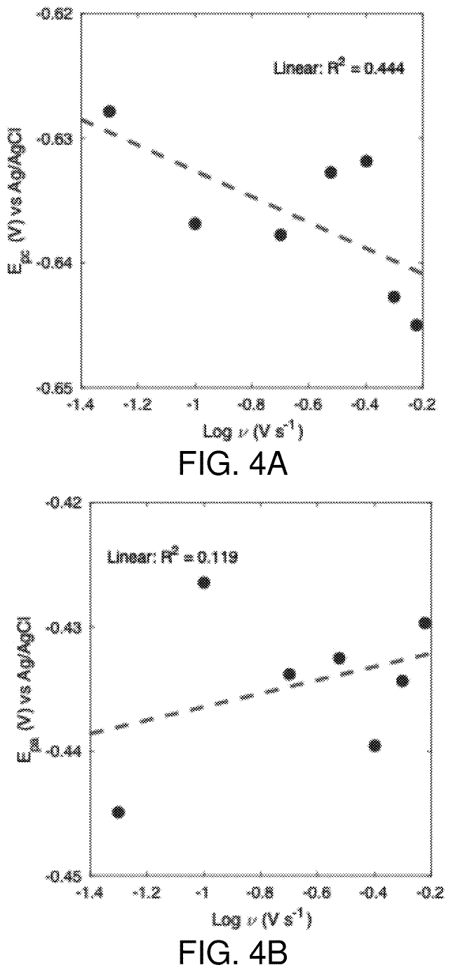

As-consumed filter coffee extracts are sufficiently conductive for direct electrochemical analysis without the addition of a supporting electrolyte (~750 Ohm resistance using a 0.0314 cm² Pt button electrode) and are self-buffered to pH of 4.8 to 5.9, depending on bean parameters and brewing water composition. Even after performing bulk electrolysis at oxygen evolution reaction (OER) potentials for two minutes, the pH of the solution remains numerically identical, indicating that coffee served as a buffer. Yet, despite the plethora of molecules in coffee extracts, the CV response for a Pt working electrode in 1.56% TDS coffee is consistent with that of acidic water (FIG. 1A). The same is true for coffee extracts at espresso strength (8% TDS-12% TDS), but the protonic feature is quenched by surface-adsorbed species so analyses on neat espresso are less pronounced, as described in more detail below. Cathodic Faradaic features map to a response expected for protonic reactions with the Pt surface (H_{UPD}), followed by H₂ evolution at more negative potentials. At positive potentials, hydroxide adsorption and eventual O₂ evolution are also evident. Scan rate was probed to ascertain whether the oxidative feature at −0.6 V is linked with the reductive protonic steps in a reversible redox couple (FIG. 2). Peak current was a linear function of square root of the scan rate for both features, which demonstrates the diffusion-controlled nature of the redox events (FIGS. 3A-3B), and the lack of an increase in the peak potential separation with increasing scan rate indicates Nernstian behavior (FIGS. 4A-4B), but the peak separation of about 200 mV at all scan rates suggests that the reversibility of the redox couple may be obfuscated by kinetic or mechanistic complications.

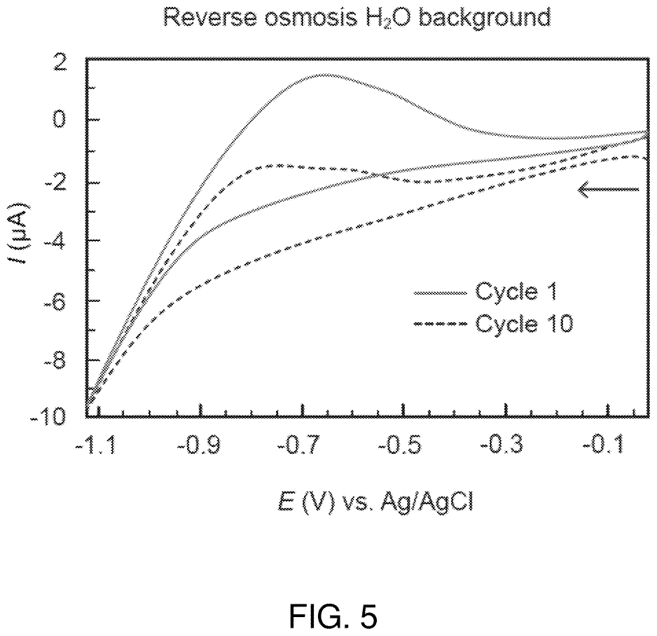

The same Pt surface sites that adsorb H⁺ and OH⁻ are also able to adsorb other molecules in solution. In the case of oxidative cycling, some impurities in water compete for the Pt surface, resulting in reduced current with subsequent cycling due to a decrease in the accessible surface area for hydrogen adsorption. A decrease in exchange current density was expected with sequential cycling, given that Pt can interact with caffeine and other molecules in coffee. When scanning from 0 V to −1.0 V, the H_{UPD} and protonic features (E=−0.4 V and −0.7 V, respectively) smear together and current decreases by about 34% from scan one to scan two, and about 18% from scan two to scan three (FIG. 1B). In water having pH=7, purified by reverse osmosis, the same features are not observed (FIG. 5), suggesting that the response is due to protonic chemical steps.

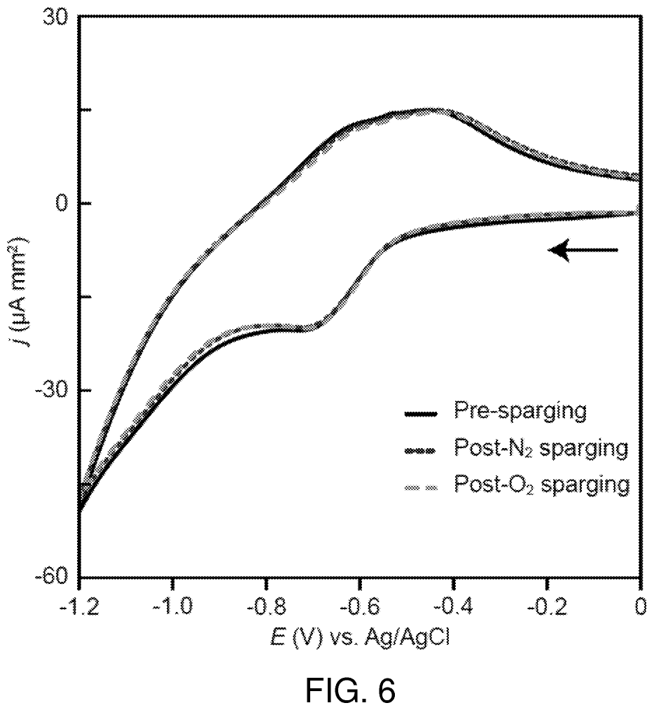

Additional examples show that these features can be mapped to H_{UPD}/weak acid oxidation and its suppression by coffee molecules, rather than fluctuations in dissolved O₂ and other spurious effects (FIG. 6). FIG. 6 is a plot of brewed coffee after one CV cycle, with and without sparging. One trace in the plot represents the brewed coffee prior to any sparging. Another trace represents a sample of coffee following 20 minutes of sparging with N₂, and another trace follows 20 minutes of sparging with O₂.

Since the integral of the current density is a function of the activity of protons, the H_{UPD} and acid features indirectly provide insights into the families of molecules in solution, which may be a function of roast, brew parameters, etc. In support of this hypothesis, H_{UPD}/acid reduction current density decreases with decreasing coffee concentration (FIG. 1C) due to the reduced number of available protons relative to more concentrated coffee. Because the feature is concentration-based, there are also fewer organic molecules competing to bind to the surface of the electrode. As described in more detail below, the integral of the charge current density of this feature linearly maps to % TDS.

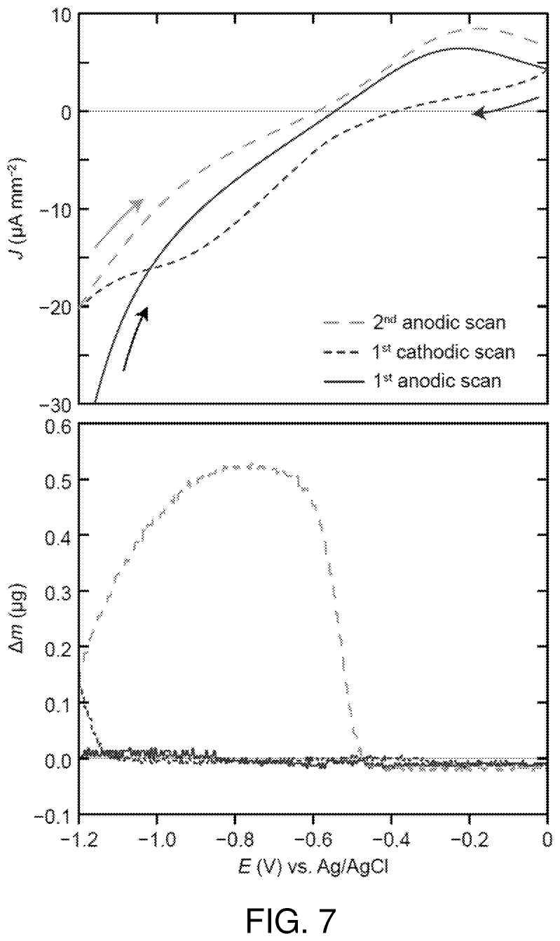

CV was performed with an electrochemical quartz crystal microbalance (QCM) with a platinum working electrode scanning at 50 mV/s (FIG. 1D). Scanning oxidatively, appreciable mass begins to accumulate on the electrode at potentials more negative than H_{UPD}—the balance is insensitive to surface hydrogen adsorption but can detect larger molecule accumulation. This delayed onset of mass can be attributed to proton-assisted adsorption of Brønsted basic species like caffeine, following a general mechanism illustrated in FIG. 1E. Voltage points indicated in FIG. 1E correspond to voltage points indicated in FIG. 1D. Upon cycling back from −1.2 V to 0 V, mass continues to accumulate until the potential falls below −0.5 V, when the electrode can liberate at least a portion of the adsorbed organic material and protons back into solution. This response is reminiscent of a kinetic trap, where the surface assembly forms in kinetically favored conditions. To determine whether the process was proton assisted, sampling was conducted from −1.2 V to 0 V (relative to Ag/AgCl), and no mass accumulation was detected (FIG. 7). Mass only began to accumulate following potentials negative of H_{UPD}, during the first reductive sweep (blue trace). These results indicate that there must be an appreciable concentration of protons on the surface before larger organic species begin to accumulate.

### Example 2—Molecular Identities of Adsorbed Material

While the total charge passed maps linearly to concentration for any particular coffee, coffees from different origins, processed in dissimilar ways, and roasted to different colors may result in differences in the emergence and suppression of the reductive features. Suppression of the convoluted redox feature at −0.55 V was studied to determine whether it is related to molecules common in coffee.

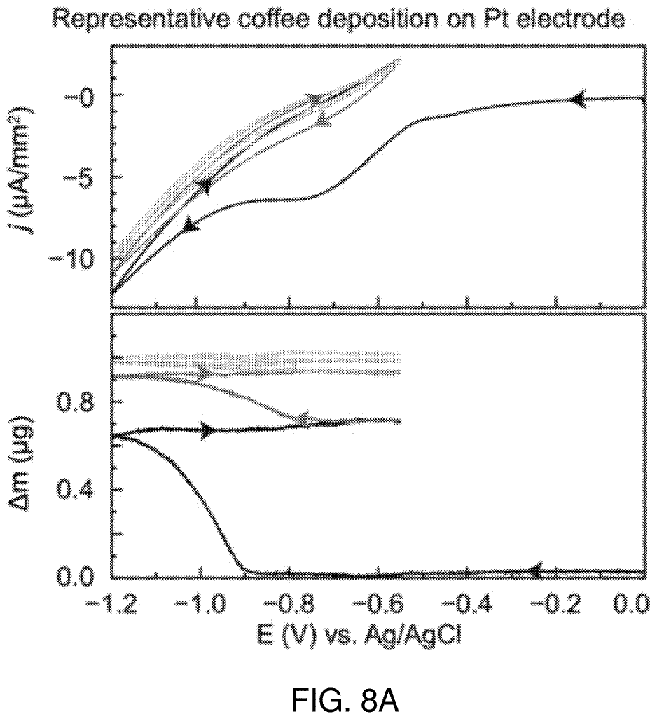

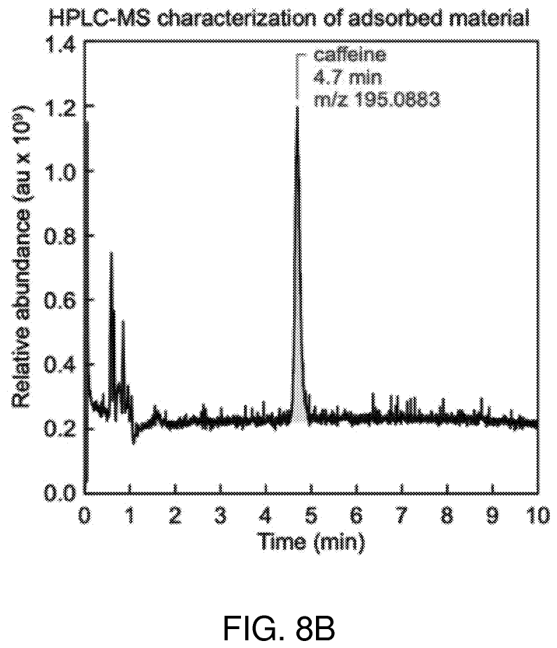

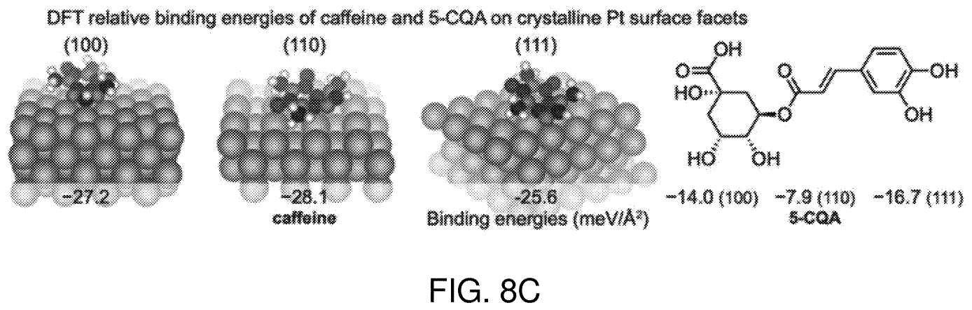

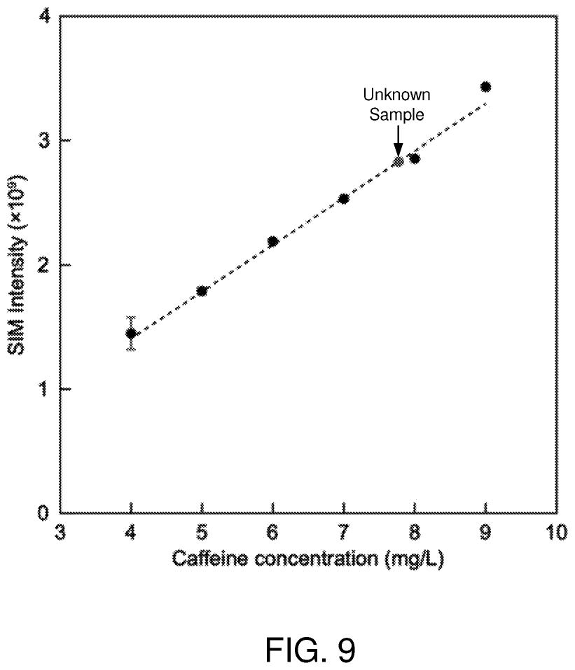

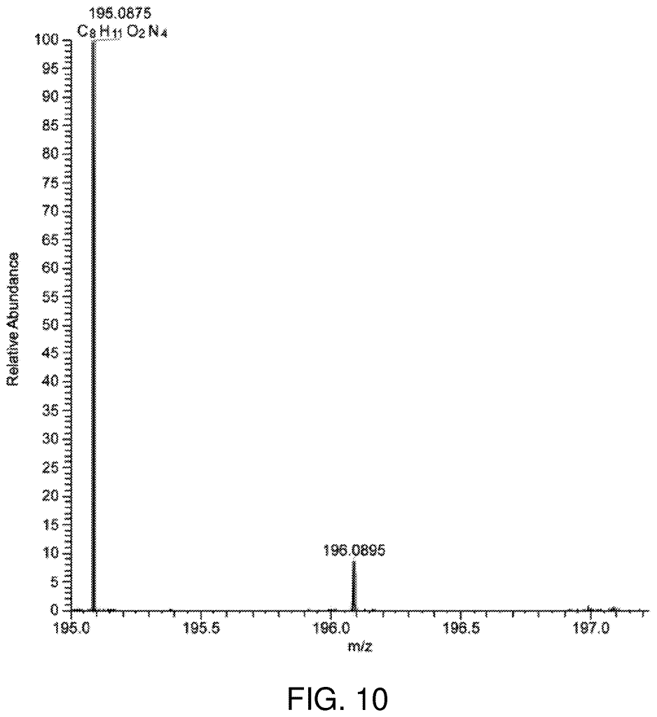

The CV cycle shown in FIG. 8A was developed to determine the molecular identities of adsorbed material. This cycle aims to promote mass accumulation on the electrode to provide enough material for characterization. Cyclic voltammetry performed at 200 mV/s sampling potentials more negative than −0.5 V, to retain accumulated mass rather than liberate the accumulated mass back into solution, per FIG. 1D. Enough adsorbed material was obtained on a Pt-mesh electrode surface by cycling 100 times from −0.55 V to −1.2 V, followed by solvating the adsorbed material in a sonicated 4 mL bath of 80:20 water:acetonitrile (v/v), and repeating four times. The adsorbates could then be separated and characterized using liquid chromatography coupled with high-resolution mass spectrometry. A calibration curve is illustrated in FIG. 9. Caffeine had adsorbed in high concentrations. The combined samples yielded 7.8±0.1 mg/kg caffeine, suggesting that the electrode gained mass from a molecule common to coffee and that the 4 mL solution contained approximately 300 μg of caffeine, or approximately 0.4% of the total caffeine in an average 6 oz cup of filter coffee (see FIGS. 9-10), or approximately 0.1% of the available caffeine in the cup. In FIG. 10, a monoisotopic peak (195.0875 m/z) is observed which matches that expected from protonated caffeine (195.08765 m/z) to a mass error of less than 1 ppm. The overlay is a simulation of the isotope pattern expected for a compound with the formula of protonated caffeine. A variety of other low concentration molecules were present but not identified, as well as some impurities derived from the plastic receptacle that the coffee was stored in.

Because other adsorbates are not in appreciable concentration to detect with high-performance liquid chromatography coupled with mass spectrometry (HPLC-MS), density functional theory molecular dynamics (ab initio MD) simulations were used to understand the adsorption enthalpies for other molecules on pristine Pt surfaces (FIG. 8C). Several geometries were obtained from the trajectories, and then DFT geometry optimizations were used to obtain equilibrium energies. The adsorption of caffeine to three stable crystal facets of Pt revealed each facet binds caffeine with a slight preference to the (110) surface. Another molecule known to be present in all green coffee is 5-CQA, with a concentration that may be strongly correlated to roast color. The adsorption of caffeine, as well as 5-CQA are both favored on all clear crystal facets of Pt, suggesting that it is likely that not only caffeine interacts with the electrode surfaces. 5-CQA has a weaker interaction than caffeine with all facets studied and a preference for the (111) surface. Interestingly, 5-CQA binds least strongly to the (110) surface, indicating that crystal facets of the Pt electrode may offer some degree of selectivity for adsorption. The models instruct that the suppression of the H_{UPD} and protonic features likely capture the ensemble of various adsorbates, providing information about the beverage concentration, as well as roast color, since roast will dictate the amount of 5-CQA (and caffeine) in the cup.

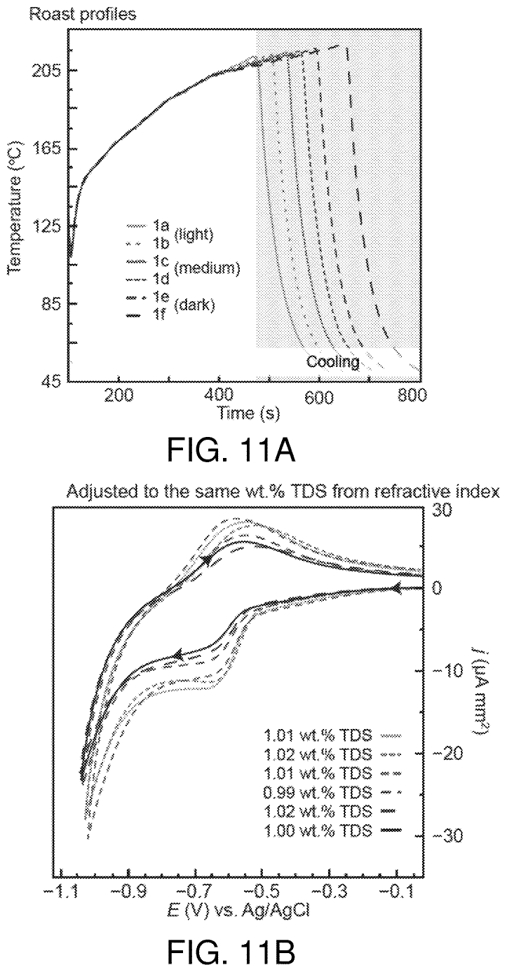

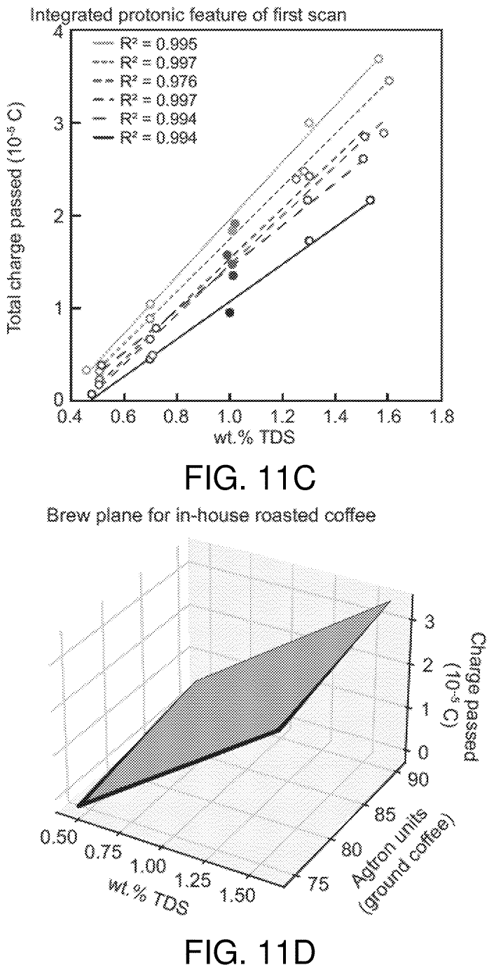

To elucidate how roast color (and subsequent chemical composition) impacts the redox response (and in particular, the reductive feature noted at approximately-0.55 V to −0.6 V), a representative specialty coffee sourced from Colombia was roasted following the profiles shown in FIG. 11A. The “light roast” profile was then systematically extended by 30 seconds and in final temperature by 2° F., to achieve four progressively darker coffees. A final sample was prepared by extending the fourth roast by a further 60 seconds and 4° F. to ensure that the roast colors covered a broad and reasonable range of values, as described in more detail below. Roast profiles were quantified using a coffee roast spectrophotometer provided by Agtron. The roast profiles yielded coffees ranging from Agtron values of 75.8 (light) to 55.7 (dark). Each sample was then rested for 7 days to allow for CO₂ offgassing, and then brewed using the Specialty Coffee Association cupping technique described in Di Donfrancesco et al. “Comparison of Results from Cupping and Descriptive Sensory Analysis of Colombian Brewed Coffee,” Sens Stud, vol. 29, no. 4, pp. 301-311 (August 2014), the relevant portion of which is herein incorporated by reference.

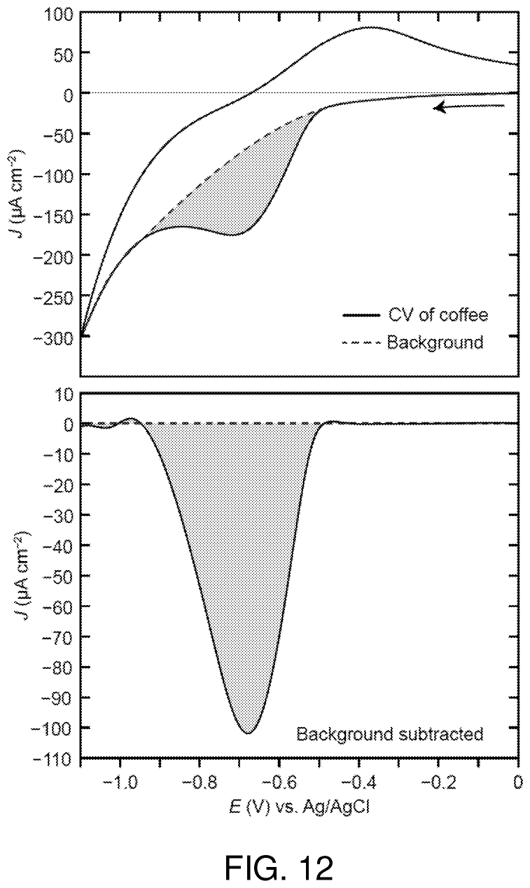

After diluting the extracts to 1.00±0.02% TDS, electrochemical differences that are a function of roast color were noted in the cyclic voltammograms shown in FIG. 11B. The lightest roast coffee passes about 50% more charge than its darker analogues. While % TDS remains fixed, the electrochemical assessment of the reductive feature is a function of roast, a parameter that impacts the sensory experience (i.e., dark roasted coffees taste “dark”). By integrating the exchange current density and adjusting for scan rate, the total charge passed can be extracted for each coffee at any arbitrary concentration (FIG. 11C). As shown in FIG. 11C, after subtracting the background contribution due to the onset of hydrogen evolution (FIG. 12), each roast shows a strong linear fit between % TDS and electrochemical charge passed. FIG. 12 shows an example of background subtraction prior to integration of the reductive feature. At top in FIG. 12, a first CV curve is shown at 1.56% TDS. At bottom, a reduction sweep is plotted after subtracting the baseline correction.

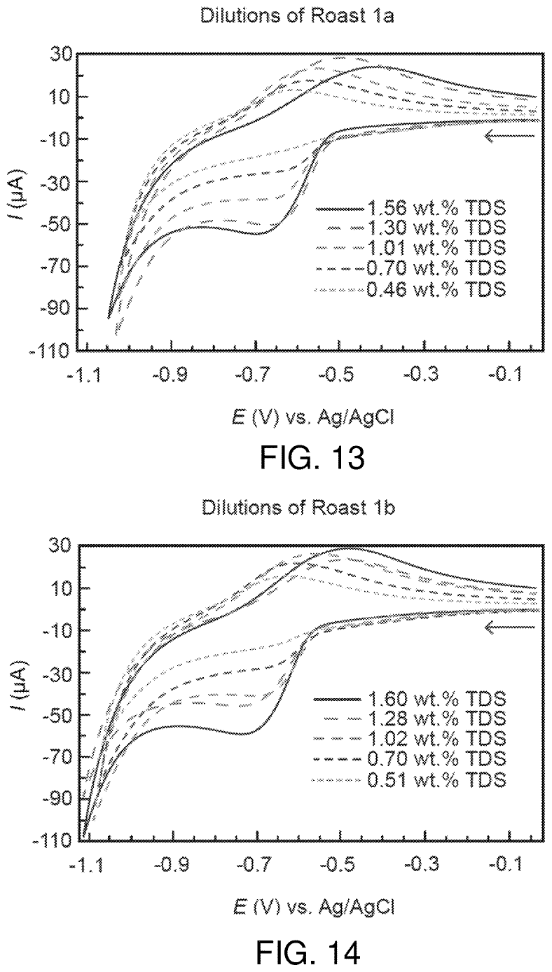

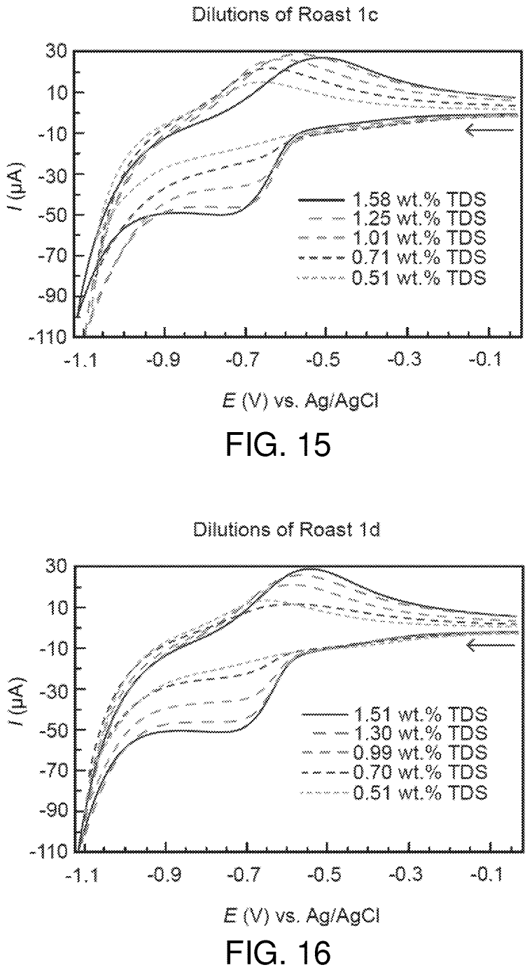

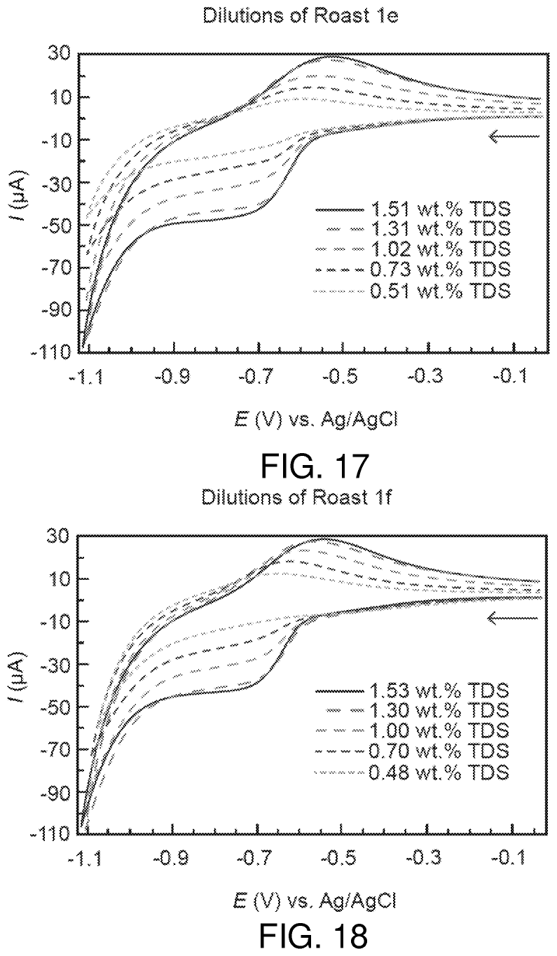

Referring again to FIG. 11C, darker roasts more effectively suppress the protonic reductive features, which may occur because they contain more oxidized molecules as a byproduct of roasting. Increasingly darker roasts yield both a shallower gradient and less total charge passed than the lighter roast counterparts. The lower current may be expected from the fact that darker coffees tend to have mildly elevated pH compared to their lighter counterparts, and that dark roasts have less water-soluble material contained within, thereby preferentially depositing on the Pt electrode. The rate of suppression of these redox waves is a function of composition (see FIGS. 13-18). Taken together, the integrated protonic feature is indirectly related to coffee composition, but it is sensitive to differences in roast-derived species.

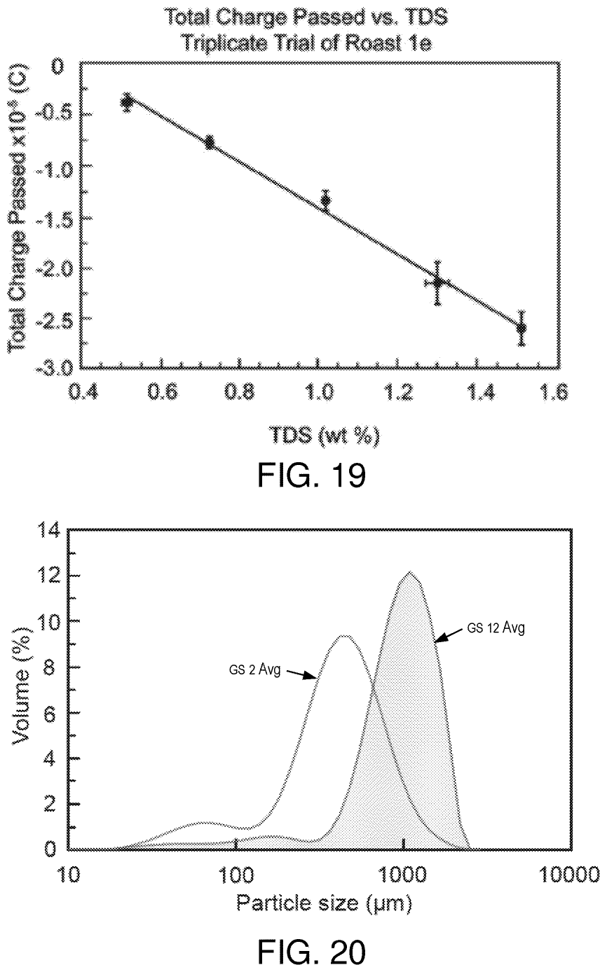

The % TDS can then be plotted along with the coffee Agtron color value and total charge passed in the H_{UPD} region, as shown in FIG. 11D. Error bars (±0.01% TDS, ±0.1×10⁻⁵ C) are omitted from FIG. 11D for clarity, but can be found in FIG. 19. While the plane plotted in FIG. 11D may be specific to the certain coffee beans used to prepare the roasted samples described herein, the fact that roast color maps linearly to current allows for a wealth of applications. For example, a series of simple CVs on progressively more dilute coffee may allow a roaster to rapidly construct a quality control calibration curve, enabling the comparison of two separate roasts. In that case, it is possible that coffees could have similar whole bean colors but different flavor profiles, or that the same coffee may have a different electrochemical response but the same bean color. In the latter case, the electrochemical response could be an indication of coffee aging, or perhaps inconsistencies in the roasting process. Additionally, the plane of FIG. 11D reveals a linear relationship with % TDS, thereby allowing for a direct measurement of % TDS using electrochemistry alone.

### Example 3—Sample Preparation

Green coffee was roasted in-house as described in the Roasting section below. These roasts canvassed industrially relevant light, medium and dark roasts (spanning whole bean Agtron values of 75.8 (lightest) to 55.7 (darkest), see Table 1).

TABLE 1
% TDS, resistance, and pH values for coffee
samples used for cyclic voltammetry.

| Roast | Sample | TDS | Resistance (Ω) | pH |
|---|---|---|---|---|
| 1a | Pure | 1.56 | 740.94 | 4.96 |
| 1a | Dil1 | 1.30 | 837.57 | 4.99 |
| 1a | Dil2 | 1.01 | 1052.93 | 5.04 |
| 1a | Dil3 | 0.70 | 1460.61 | 5.12 |
| 1a | Dil4 | 0.46 | 2091.03 | 5.24 |
| 1b | Pure | 1.60 | 733.99 | 5.06 |
| 1b | Dil1 | 1.28 | 875.55 | 5.08 |
| 1b | Dil2 | 1.02 | 1048.32 | 5.12 |
| 1b | Dil3 | 0.70 | 1425.42 | 5.19 |
| 1b | Dil4 | 0.51 | 1977.29 | 5.25 |
| 1c | Pure | 1.58 | 719.85 | 5.12 |
| 1c | Dil1 | 1.25 | 835.85 | 5.13 |
| 1c | Dil2 | 1.01 | 1048.65 | 5.17 |
| 1c | Dil3 | 0.71 | 1445.79 | 5.23 |
| 1c | Dil4 | 0.51 | 1922.64 | 5.31 |
| 1d | Pure | 1.51 | 768.58 | 5.17 |
| 1d | Dil1 | 1.30 | 876.40 | 5.19 |
| 1d | Dil2 | 0.99 | 1103.23 | 5.23 |
| 1d | Dil3 | 0.70 | 1507.73 | 5.30 |
| 1d | Dil4 | 0.51 | 1994.35 | 5.38 |
| 1e Run 1 | Pure | 1.50 | 789.60 | 5.30 |
| 1e Run 1 | Dil1 | 1.26 | 861.16 | 5.29 |
| 1e Run 1 | Dil2 | 1.01 | 1091.62 | 5.35 |
| 1e Run 1 | Dil3 | 0.72 | 1491.01 | 5.42 |
| 1e Run 1 | Dil4 | 0.51 | 1992.82 | 5.50 |
| 1e Run 2 | Pure | 1.50 | 786.07 | 5.30 |
| 1e Run 2 | Dil1 | 1.31 | 865.86 | 5.29 |
| 1e Run 2 | Dil2 | 1.01 | 1090.67 | 5.36 |
| 1e Run 2 | Dil3 | 0.72 | 1495.26 | 5.42 |
| 1e Run 2 | Dil4 | 0.53 | 1980.11 | 5.50 |
| 1e Run 3 | Pure | 1.51 | 771.35 | 5.30 |
| 1e Run 3 | Dil1 | 1.31 | 872.56 | 5.29 |
| 1e Run 3 | Dil2 | 1.02 | 1091.54 | 5.36 |
| 1e Run 3 | Dil3 | 0.73 | 1489.70 | 5.42 |
| 1e Run 3 | Dil4 | 0.51 | 1990.33 | 5.50 |
| 1f | Pure | 1.53 | 783.20 | 5.38 |
| 1f | Dil1 | 1.30 | 860.28 | 5.39 |
| 1f | Dil2 | 1.00 | 1077.01 | 5.44 |
| 1f | Dil3 | 0.70 | 1465.19 | 5.52 |

The coffee was allowed to rest for three days before being ground with a Mahlkönig EK-43 grinder (particle size distributions are presented in FIG. 20) and brewed via cupping method with a brew ratio of 1:13.5 using 93° C. water (tap water provided by the city of Eugene, OR and filtered using a PENTAIR EVERPURE CONSERV 75E Reverse Osmosis system with remineralization to 30 mg/L CaCO₃). Grinder setting “2” was used for ground bean color assessment, while grinder setting “12” was used for cupping coffee brews. The coffee was allowed to contact the water for 4 minutes, without agitation or stirring. The samples were then filtered through paper and allowed to come to room temperature (21.6° C.). The samples were stirred before each aliquot was taken to ensure homogeneity. The cumulative concentration of solvated coffee material (total dissolved solids as a mass percentage) was calculated from the measured refractive index.

### Example 4—CV Measurements

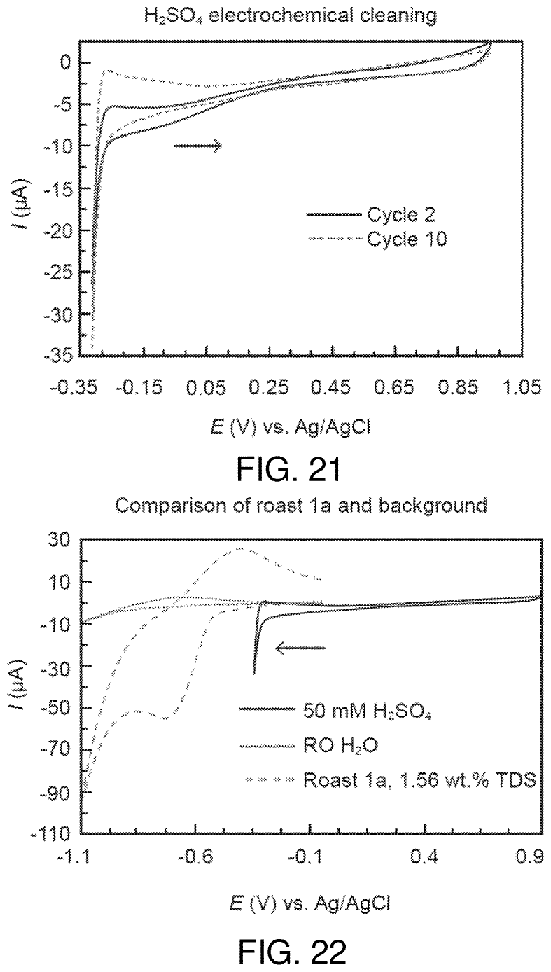

In this example, a three-electrode electrochemical setup was employed for all voltammetric measurements. CV was performed using a GAMRY 1010B potentiostat controlling Pt working and counter electrodes, and an Ag/AgCl (sat. KCl) reference electrode, purchased from CH Instruments. Prior to data collection, the working and counter electrodes were polished (10 scans at 300 mV/s scan rate in 50 mM H₂SO₄, until key H₂SO₄ redox features overlaid, see FIG. 21). Signal-to-noise was then assessed through comparison between the brew water, the cleaning solution, and the coffee samples, as shown in FIG. 22. FIG. 22 shows an overlay of Roast 1a at 1.56% TDS with a background scan of the reverse osmosis H₂O used for coffee brewing and 50 mM solution of H₂SO₄ used for electrochemical polishing of electrodes. Scan rates for H₂O and coffee were 200 mV/s and 300 mV/s for H₂SO₄. Measurements were made directly on as-brewed coffee, with no supporting electrolyte or buffer. The bulk resistivities in all samples were found to be between 100-1000Ω and the pH of each solution is noted in Table 1 (above). Since coffee can be a solution of weak acids, the solutions are self-buffering to a pH of approximately 5.0.

### Example 5—Particle Size Distribution

Ground coffee particles prepared as per Example 3 were measured using a MALVERN MASTERSIZER 2000 laser diffraction system. Solid samples were dispersed into an airstream (2.0 bar) of compressed breathing-quality air and passed through a He—Ne laser beam at 633 nm and a solid-state blue light laser beam at 466 nm. The particles interfere with and scatter light at different angles based on their sizes. The scattered light is then detected and analyzed to give the particle size distribution of the sample. Ground coffee samples were run in triplicate and the average particle size distributions are plotted in FIG. 20.

### Example 6—QCM

A QCM200 instrument equipped with a crystal holder was used to perform all QCM experiments. Pt-coated liquid-plating 5 MHz quartz crystal electrodes with a Ti adhesion layer provided by FIL-TECH were used. The crystal holder ensures that only one side of the Pt-coated electrode is accessible to the solution and the controller reads the associated frequency. A BIOLOGIC SP-300 potentiostat with a Pt wire counter electrode and Ag/AgCl (sat. KCl) reference electrode was used for collecting CV data corresponding with QCM data. The QCM frequency data was converted to mass values via the Sauerbrey equation as follows:

$$Δ f = \frac{- {C}_{f} Δ m}{A} \qquad ( 1 )$$

In Equation (1), f is the frequency as read from the QCM controller, C_{f} is the calibration constant which is given to be 56.6 Hz·cm²/ug for the FIL-TECH electrodes, and A is the electrode surface area which is 0.4987 cm².

### Example 7—High Resolution Mass Spectrometry

To accumulate mass on the electrode surface for further analysis, a Pt mesh electrode from BASI RESEARCH PRODUCTS with a surface area of five cm² was used as the working electrode with a Pt wire counter electrode and an Ag/AgCl (sat. KCl) reference electrode. The CV scan window was shortened to avoid the oxidation wave where the accumulated mass desorbed from the electrode surface. CV was run in as-brewed coffee with about 1.5% TDS for 50-100 cycles. Immediately upon completion of the CV, the working electrode was removed from the sample, patted dry with a wipe, and submerged into a solution of 80% 18 MΩ·cm water and 20% HPLC grade acetonitrile (SIGMA-ALDRICH) (v/v). The solution and working electrode were sonicated for 10 minutes and then the electrode was removed and rinsed. This process was repeated a total of four times to ensure that the solution contained enough redissolved residue for mass spectrometry analysis.

A THERMO SCIENTIFIC Vanquish chromatograph paired with a Q-EXACTIVE tandem orbitrap mass spectrometer, operating in positive polarity mode with a nominal resolution of 70,000, was used to identify unknown compounds and to quantify caffeine in the residue sample. An ACE EXCEL 2 SUPERC18 column (100 mm×2.10 mm, 2 μm) was used to achieve separation of the compounds in the residue using a mobile phase consisting of: ultrapure water, methanol (HONEYWELL), acetonitrile (JT BAKER), and 1M acetic acid. All mobile phase components also contained 0.1% formic acid. The liquid flow rate was 350 μL/min, and the gradient used for the compound identification runs can be found in Table 2.

TABLE 2
Liquid chromatography mobile phase gradient for compound
identification analysis. All transitions are linear.

| Time | Water | Methanol | Acetonitrile | 1M Acetic |
|---|---|---|---|---|
| (min) | (%) | (%) | (%) | Acid (%) |
| 0 | 67 | 8 | 5 | 20 |
| 4.5 | 63 | 12 | 5 | 20 |
| 28 | 0 | 55 | 40 | 5 |
| 35 | 0 | 55 | 40 | 5 |
| 40 (end) | 67 | 8 | 5 | 20 |

The MS system was run in full MS mode (over a range of 150-1000 m/z) for compound identification. Identification of caffeine was confirmed by investigating the TIC peak located at the same retention time as that of a caffeine standard solution and matching the highest intensity peak in the mass spectrum at that retention time to that of protonated caffeine to less than 2 ppm of mass error. Further, the observed isotope pattern agreed with a simulation of an expected pattern for caffeine generated using the Freestyle data analysis software package (THERMO SCIENTIFIC), FIG. 10.

Quantification of caffeine was performed using targeted selected ion monitoring (t-SIM) mode centered at 195.0876 m/z with an isolation width of 1.0 m/z. The unknown, and each standard, was measured in triplicate and the data processed using an automated peak-finding and peak-fitting algorithm available in the FREESTYLE data analysis application. Each sample was injected into the column and a short mobile phase gradient was used with a total run time of 8 minutes per injection. The details of this gradient can be found in Table 3.

TABLE 3
Liquid chromatography mobile phase linear gradient for
caffeine quantification. All transitions are linear.

| Time | Water | Methanol | Acetonitrile | 1M Acetic |
|---|---|---|---|---|
| (min) | (%) | (%) | (%) | Acid (%) |
| 0 | 67 | 8 | 5 | 20 |
| 8 | 63 | 12 | 5 | 20 |

### Example 8—Roasting

A green coffee, El Tambo from Cauca, Colombia, was sourced from TAILORED COFFEE ROASTERS of Eugene, OR. This coffee is representative of an average specialty coffee, and costs approximately $10/kg. The coffee was roasted in 50 g sample batches using an IKAWA PRO50. Sample batches were roasted using a simple roast profile and the associated profiles are presented in FIG. 11A. The initial roast profile (roast 1a) was roasted for 6 minutes and 17 seconds and ended at 412° F. Additional roasts were each extended by an additional 30 seconds and 2° F. for a total of 8 minutes and 17 seconds and an ending temperature of 420° F. for roast 1e. The roast extension was doubled to 60 seconds and 4° F. between roasts 1e and 1f to ensure a suitably dark sample was included. Roast colors are presented on the Agtron Gourmet scale, where Agtron values range from 0 (black) to 150 (green, unroasted). The values were measured using the coffee dipper spectrophotometer in whole bean mode. The data are collated in Table 4.

TABLE 4
Roasting conditions and their associated Agtron numbers.
Grind setting “2” was used on an EK-43 grinder
to obtain the ground Agtron values.

| | Total Time | Final Temperature | Whole Bean | Ground Coffee |
|---|---|---|---|---|
| Roast | (min) | (° F) | Agtron | Agtron |
| 1a | 6:17 | 412 | 75.8 | 90.3 |
| 1b | 6:47 | 414 | 72.2 | 88.8 |
| 1c | 7:17 | 416 | 64.3 | 83.6 |
| 1d | 7:47 | 418 | 61.2 | 80.3 |
| 1e | 8:17 | 420 | 58.2 | 74.6 |
| 1f | 9:17 | 424 | 55.7 | 76.1 |

### Example 9—Computational Models

All calculations were performed using the Vienna Ab initio Simulation Package (VASP 5.4.4) using a plane wave basis set and projector augmented-wave pseudopotentials. Bare surfaces of Pt(100), Pt(110), and Pt(111) as well as gas-phase simulations of caffeine and chlorogenic acid were geometrically optimized with density functional theory using a cutoff energy of 400 eV, the PBEsol functional, and a k-point spacing of 0.03 2π/Å with a 20 Å vacuum layer. Interlayer Van der Waals dispersion interactions were modeled using the DFT+D3 formalism with Becke-Johnson damping. The unit cells of Pt (hkl) contained four discrete layers of Pt atoms and the ionic coordinates of the bottom two layers were frozen while the top two layers were allowed to relax. Density functional theory optimizations of caffeine/Pt (hkl) and chlorogenic acid/Pt (hkl) were initialized from minimum energy configurations derived from molecular dynamics simulations and used a single k-point at Gamma. For caffeine/Pt (hkl), four layers of Pt were used with the bottom two layers frozen, and for chlorogenic acid/Pt (hkl), two layers of Pt were used with the bottom layer frozen. Molecular dynamics simulations were performed with a 300 eV cutoff energy using the Nose-Hoover thermostat, with 1 fs time steps beginning at 300 K and approaching 1 K, based on glycerol adsorption at Pt surfaces. For both DFT and MD, the convergence criteria were 10 meV/Å for atomic forces and 1×10⁻⁶ eV for total energy.

### Example 10—Preparation of Coffee Samples

In this example, espresso was prepared using a NUOVA SIMONELLI MYTHOS ONE CLIMA PRO grinder and a VICTORIA ARDUINO BLACK EAGLE espresso machine set to a brewing temperature of 94° C. and 8 bar of pump pressure. The grind size was adjusted to yield 40 g of espresso from 18 g of ground coffee (Red Brick espresso blend, Square Mile Coffee Roasters, London, UK) in 30±2 s. Any shots that fell out of the time range were rejected, and espresso was prepared until 60 acceptable shots were collected. Before electrochemical treatment, the TDS of each shot was measured using a VST refractometer and a small aliquot of espresso filtered through a 0.45 μm PTFE syringe filter (mean=9.05%, SD=0.19%).

### Example 11—Electrochemical Modification of Coffee Samples

Espresso shots prepared as per example 10 were randomly divided into 12 groups of 5 shots, which included one unmodified control sample and four samples, out of which one sample would be subjected to each of the different electrochemical treatment procedures.

Electrochemical treatments were applied to brewed espresso using a Gamry Interface 1010B potentiostat in a 3-electrode configuration with a Pt mesh working electrode, Pt wire counter electrode, and Ag/AgCl (sat. KCl) reference electrode. Chronoamperometry routines were performed at +350 mV and +700 mV for 30 s and 120 s with 300 RPM magnetic stirring.

Following electrolysis, all samples were diluted with the same water used for brewing to a target americano-strength TDS of 1.30% (mean=1.30%, SD=0.03%) and stored in sealed glass bottles in a 4° C. refrigerator overnight.

### Example 12—Sensory Evaluation

After overnight storage, the samples prepared as per example 11 were served to a panel of 57 trained tasters using the Specialty Coffee Association (SCA) Coffee Value Assessment (CVA) system after being trained on its use. The SCA CVA system is described in “A System to Assess Coffee Value: Understanding the SCA's Coffee Value Assessment” published June 2024, the relevant portion of which is incorporated herein by reference.

Each panelist was served a tray with five small paper cups placed in a straight line across the tray and labeled with a randomized three-digit code. The leftmost sample was the unmodified control whereas the order of the treated samples was randomized, and the panelists were instructed to taste from left to right before silently filling out a standardized form provided for the SCA CVA. The form contained continuous line scales where the panelist marked a score on a scale from 0 to 15 for overall quality and the intensities of aroma, flavor, aftertaste, acidity, sweetness, and mouthfeel. Additionally, boxes that list options for common aromas, flavors, main tastes, acidity types, and mouthfeels are available. The continuous scores were digitized by measuring distance (in mm) from 0 to the center of the mark applied by the panelist. The identity of the samples, the purpose of the experiment, and the mechanism of treatment were not revealed to the panelists until the conclusion of the assessment.

Statistical analysis of the sensory data was performed using “XLSTAT”. A discriminant analysis (DA) of the digitized intensity line scale scores was carried out to model 95% confidence interval centroids for each sample group and to assign the variables that contribute to the position of the group along the principal component axes. The DA was performed for the intensity line scales for five samples evaluated by 57 judges. The descriptors of the line scale were: overall quality, aroma, flavor, aftertaste, acidity, sweetness, and mouthfeel.

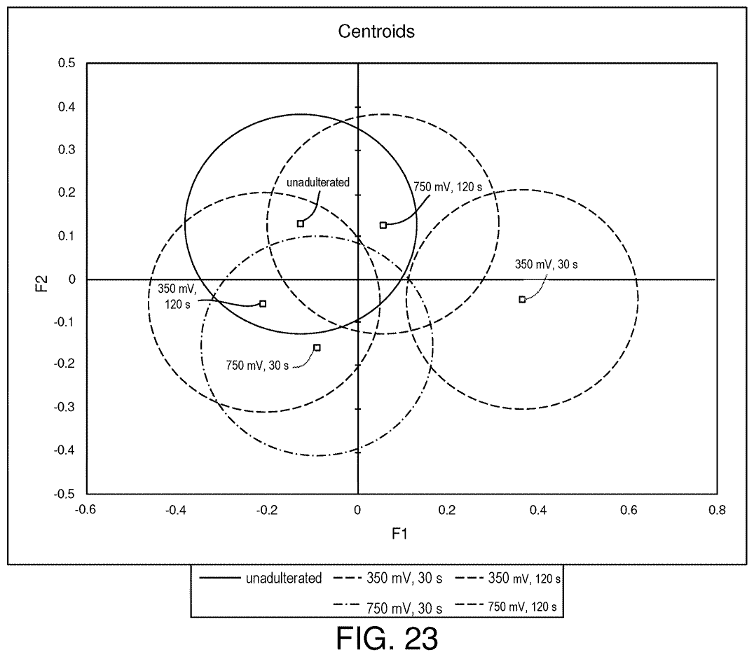

The results of the DA with 95% confidence intervals separated the coffee samples into five distinct groups based on the treatment (FIG. 23). There were statistical differences among the samples: the samples treated at +350 mV for 30 s are significantly different from the unadulterated samples and the samples treated at +350 mV for 120 seconds. Some samples (such as those treated at +700 mV/120 s and +700 mV/30 s) are not significantly different.

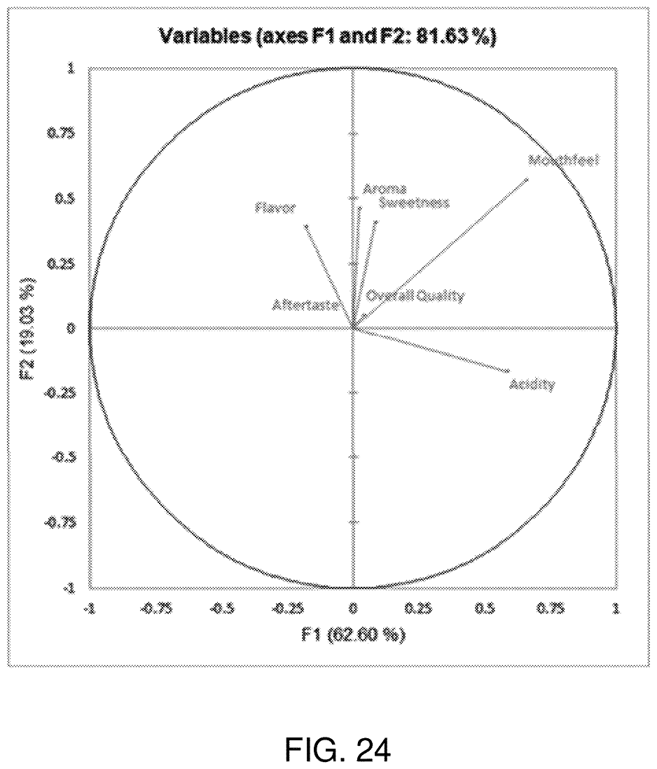

Two statistically significant discriminant functions explained 81.63% of the variability, with F1 and F2, contributing 62.60% and 19.03%, respectively (FIG. 24). Additional functions did not add to the total variance significantly (data not shown). Coffee labeled “moderately adulterated +” was treated at +350 mV for 30 s and had the highest positive score in the F1 direction with a profile of higher acidity and mouthfeel (FIGS. 23-24). Coffee labeled “a lot adulterated ++” was treated at +750 mV for 120 s and showed a more intense mouthfeel, followed by aroma and sweetness. Coffee labeled “a lot adulterated ++” and “moderately adulterated +” are characterized by overall quality. Unadulterated coffee showed a more intense flavor and aftertaste. Coffee labeled “a lot adulterated +” was treated at +350 mV for 30 s and coffee labeled “moderately adulterated ++” was treated at +750 mV for 30 s. Both of these coffees (“a lot adulterated +”=+350 mV/30 s, and “moderately adulterated ++”=+750 mV/30 s) had similar profiles.

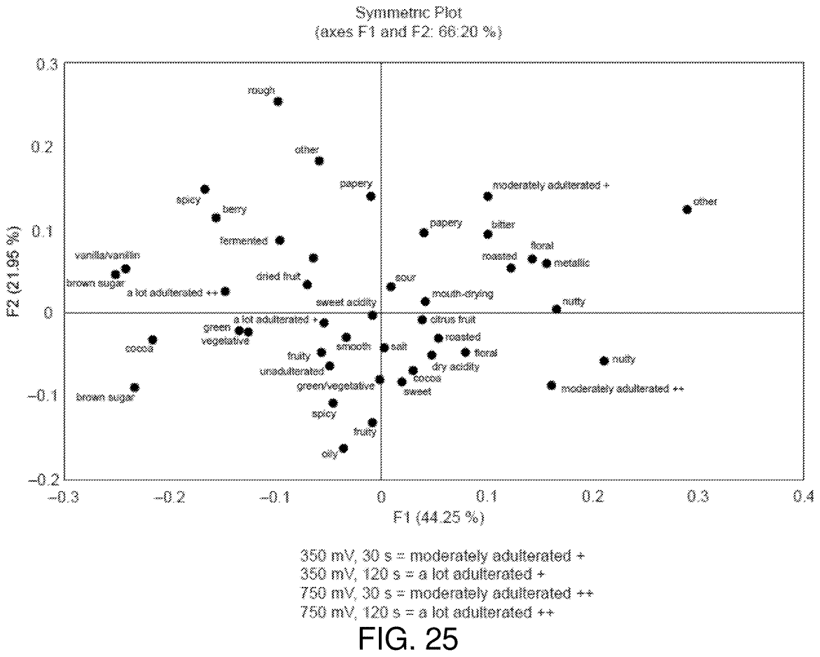

Data for aroma, flavor, main tastes, acidity, and mouthfeel descriptors show variations in terms associated with coffee treatments (FIG. 25). Across the first two dimensions, a 66.2% variance was found. Coffee labeled “a lot adulterated +” (±350 mV/120 s) and unadulterated coffees were described with profiles of smooth, fruity, green/vegetative, and sweet acidity. Coffee labeled “a lot adulterated ++” (±700 mV/120 s) was described with notes of brown sugar, vanilla, and dried fruits. Coffee labeled “moderately adulterated +” (±350 mV/30 s) was described as bitter, papery, roasted, floral, metallic, and coffee labeled as “moderately adulterated ++” (±700 mV/30 s) was described as nutty.

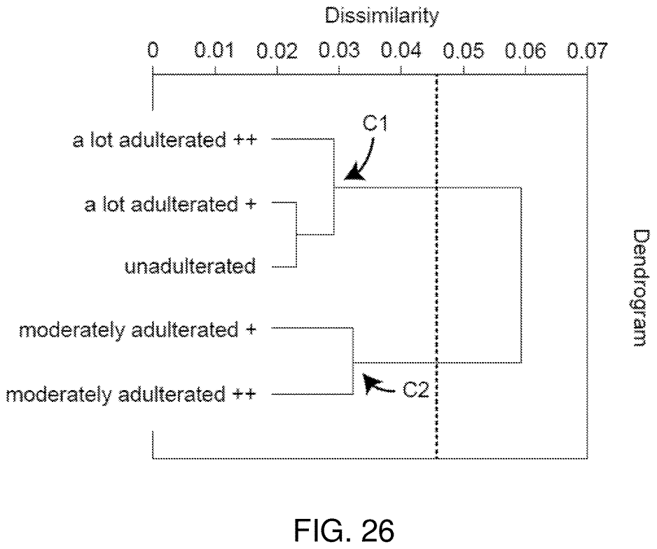

FIG. 26 shows a dendrogram from hierarchical clustering analysis. Clustering revealed two significantly different distinct clusters, with C1 being the cluster of unadulterated coffee, coffee treated with +350 mV/120 s, and coffee treated with +700 mV/120 s, and C2 being the cluster of coffee treated with +350 mV/30 s and coffee treated with +700 mV/30 s.

Additional analysis was performed by converting the raw digitized line scale scores into rankings for each panelist, from 1 (most intense/best) to 5 (least intense/worst). The sample groups were assessed for statistical difference using a two-sample t-test where the average ranking of the unmodified control was compared to the most different average ranking of the four modified samples for each characteristic. Results of this analysis are provided in Table 5.

TABLE 5
Panelist rankings of electrochemically modified coffee.

| | Treatment | | | | | |
|---|---|---|---|---|---|---|
| | | +350 mV/ | +350 mV/ | +700 mV/ | +700 mV/ | |
| Rank | Unmodified | 30 s | 120 s | 30 s | 120 s | p-value |
| Quality | 2.60 | 2.89 | 2.83 | 3.20 | 2.64 | 0.012 |
| Acidity | 3.10 | 2.64 | 2.92 | 3.08 | 2.79 | 0.066 |
| Sweetness | 2.97 | 2.83 | 2.85 | 3.19 | 2.64 | 0.018 |

The panel generally showed that espresso can be electrochemically altered to improve perceived acidity and sweetness, which are desirable attributes in specialty coffee. This result demonstrates a new mechanism for affecting the quality of coffee and may be employed to improve or alter any coffee using simple electrochemical strategies.

### Example 13—HPLC Analysis of Electrochemically Modified Coffee

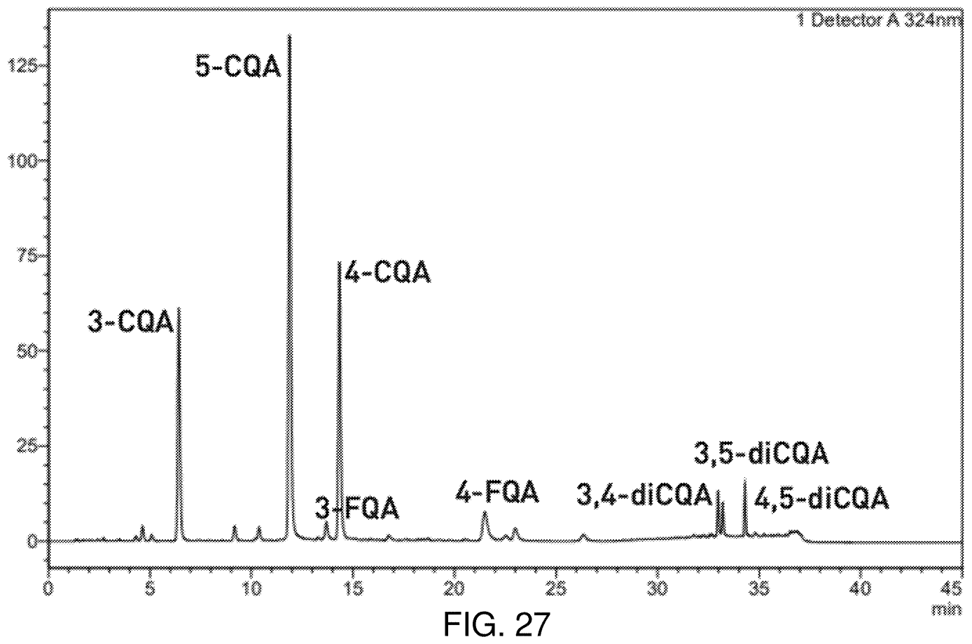

HPLC was performed with a Shimadzu Prominence-i LC-2030 Plus HPLC system using the method described in Jeon, J.-S.; Kim, H.-T.; Jeong, I.-H.; Hong, S.-R.; Oh, M.-S.; Park, K.-H.; Shim, J.-H.; Abd El-Aty, A. M. Determination of Chlorogenic Acids and Caffeine in Homemade Brewed Coffee Prepared under Various Conditions. J. Chromatogr. B (2017), 1064, 115-123, the entirety of which is herein incorporated by reference in its entirety for all purposes. Briefly, 0.5 mL of brewed coffee was clarified with Carrez solutions I and II, diluted to 10 mL with 10% MeOH, and centrifuged. The supernatant was filtered through a 0.45 μm PTFE syringe filter and injected onto a 150×4.6 mm ACCUCORE 2.6 μm C18 column maintained at 40° C. in a gradient of 20 mM KH₂PO₄±0.1% H₃PO₄ and acetonitrile+0.1% H₃PO₄. The peaks measured at 324 nm were identified by their elution order according to the assignments made in Jeon et al. (supra) and are shown in FIG. 27. Integrated results are provided in Table 6, which reveals substantial conversion of 3,5-diCQA.

TABLE 6
HPLC analysis of electrochemically modified coffee.

| | | +350 mV/ | +350 mV/ | +700 mV/ | +700 mV/ | |
|---|---|---|---|---|---|---|
| % area | Unmodified | 30 s | 120 s | 30 s | 120 s | Conversion |
| 3-CQA | 19.1 | 19.6 | 19.6 | 19.8 | 19.6 | 3% |
| 5-CQA | 43.7 | 43.9 | 44.0 | 43.7 | 43.8 | 1% |
| 3-FQA | 1.68 | 1.62 | 1.63 | 1.64 | 1.63 | 4% |
| 4-CQA | 23.6 | 23.8 | 23.8 | 23.7 | 23.7 | 1% |
| 4-FQA | 5.15 | 4.94 | 4.86 | 5.08 | 5.06 | 6% |
| 3,4-diCQA | 2.23 | 2.15 | 2.15 | 2.13 | 2.08 | 7% |
| 3,5-diCQA | 2.01 | 1.66 | 1.66 | 1.65 | 1.57 | 22% |
| 4,5-diCQA | 2.52 | 2.33 | 2.32 | 2.34 | 2.48 | 7% |

### Example 14—Flavor Evolution Over Time in Coffee

Samples of a dark roast, a medium roast, and a light roast coffee were brewed as described above according to Example 10 using 16 g of ground coffee per sample. The samples were subjected to one of four different potentials (−2 V, −1 V, +1 V, and +2 V, all relative to Ag/AgCl (sat. KCl)).

Electrochemical treatments were applied to the samples using a GAMRY Interface 1010B potentiostat in a 3-electrode configuration with a graphite rod working electrode and Pt wire counter electrode. The following operational parameters were used for all treatments: 10 Hz sampling frequency, 10 nA current range, 1 μC charge resolution.

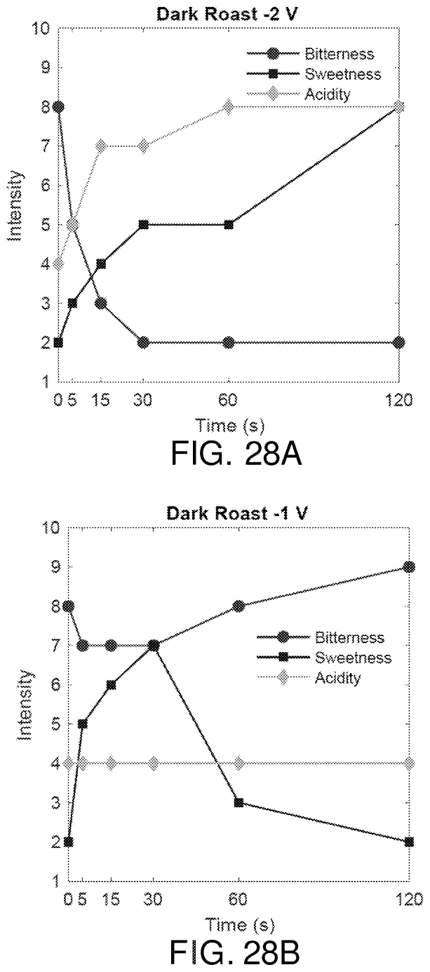

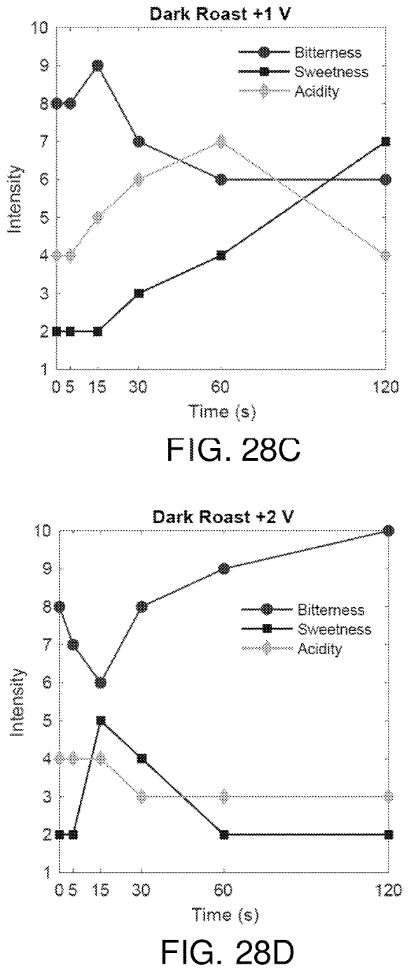

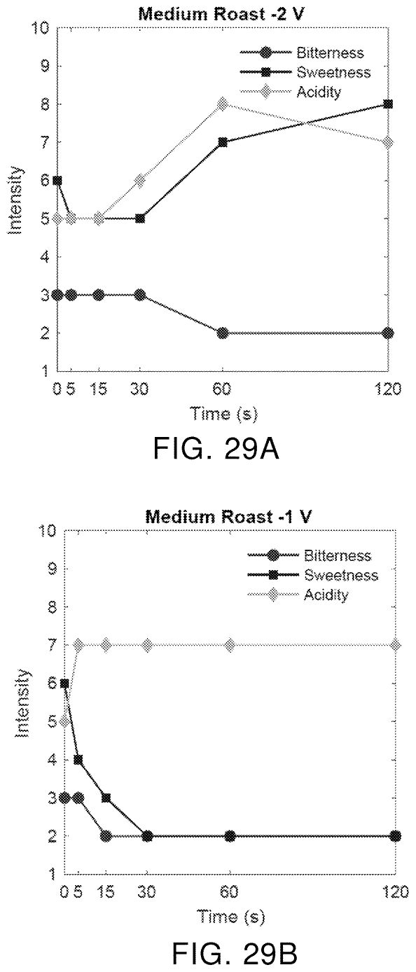

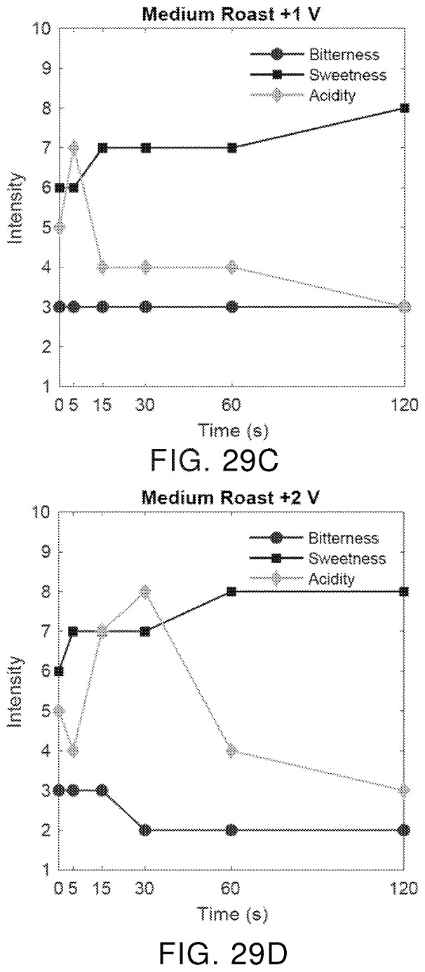

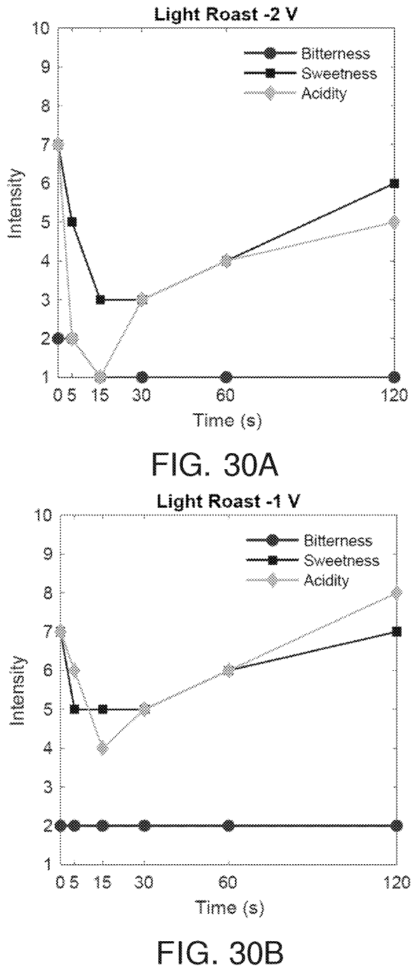

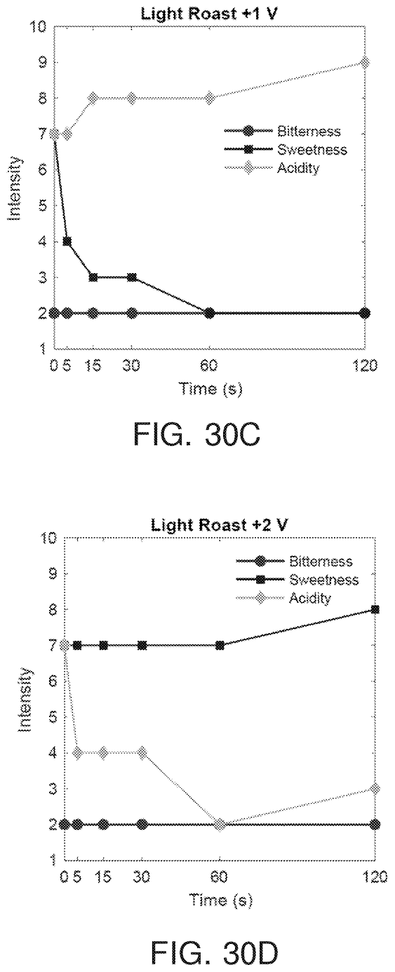

FIGS. 28A-28D are plots of flavor evolution over time for the dark roast coffee samples over time at each of the four different voltages (−2 V, −1 V, +1 V, and +2 V for FIGS. 28A-28D, respectively). FIGS. 29A-29D represent the same sequences of flavor evolution over time for the medium roast coffee samples, and FIGS. 30A-30D represent the light roast coffee samples. The samples were treated electrochemically and tasted at different time points to assess how the flavors change as a function of treatment time/charge passed at specific voltages and as a function of the roast level of the coffee. The three major flavors assessed (bitterness, sweetness, and acidity) represent the relevant sensory attributes of the coffees.

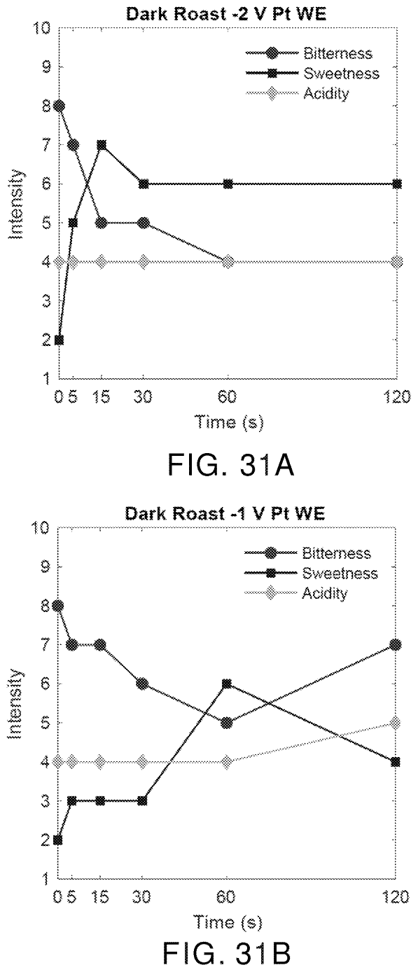

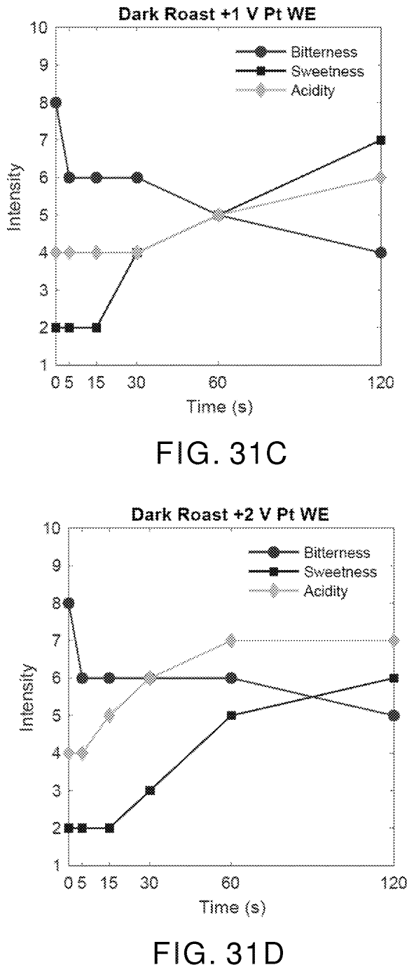

Electrochemical treatments were also applied to another set of dark roast coffee samples using a 3-electrode configuration with a Pt mesh working electrode and a Pt wire counter electrode. The same operational parameters were used (10 Hz sampling frequency, 10 nA current range, 1 μC charge resolution). FIGS. 31A-31D are plots of flavor evolution over time at each of four different voltages (−2 V, −1 V, +1 V, and +2 V for FIGS. 31A-31D, respectively, all relative to Ag/AgCl (sat. KCl)) for the dark roast samples treated using the Pt mesh working electrode and the Pt wire counter electrode.

Table 7 summarizes the charge passed to the coffee at each tasting point. As an illustrative example, the charge passed at 100 ms for the light roast coffee sample is also reported.

TABLE 7
Charge passed through each sample.

| | | | Time | Charge |
|---|---|---|---|---|
| Roast | Working electrode | Voltage | (s) | (mC) |
| Dark | Graphite rod | −2 | 5 | −818.1 |
| Dark | Graphite rod | +2 | 5 | 569.8 |
| Dark | Graphite rod | −1 | 5 | −599.0 |
| Dark | Graphite rod | +1 | 5 | 100.5 |
| Dark | Graphite rod | −2 | 15 | −2224 |
| Dark | Graphite rod | +2 | 15 | 1164 |
| Dark | Graphite rod | −1 | 15 | −934.5 |
| Dark | Graphite rod | +1 | 15 | 178.9 |
| Dark | Graphite rod | −2 | 30 | −3921 |
| Dark | Graphite rod | +2 | 30 | 1864 |
| Dark | Graphite rod | −1 | 30 | −1124 |
| Dark | Graphite rod | +1 | 30 | 259.0 |
| Dark | Graphite rod | −2 | 60 | −7112 |
| Dark | Graphite rod | +2 | 60 | 3035 |
| Dark | Graphite rod | −1 | 60 | −1343 |
| Dark | Graphite rod | +1 | 60 | 375.9 |
| Dark | Graphite rod | −2 | 120 | −13320 |
| Dark | Graphite rod | +2 | 120 | 5096 |
| Dark | Graphite rod | −1 | 120 | −1627 |
| Dark | Graphite rod | +1 | 120 | 546.6 |
| Dark | Pt mesh | −2 | 5 | −231.5 |
| Dark | Pt mesh | +2 | 5 | 77.62 |
| Dark | Pt mesh | −1 | 5 | −59.75 |
| Dark | Pt mesh | +1 | 5 | 33.28 |
| Dark | Pt mesh | −2 | 15 | −625.1 |
| Dark | Pt mesh | +2 | 15 | 174.5 |
| Dark | Pt mesh | −1 | 15 | −128.5 |
| Dark | Pt mesh | +1 | 15 | 54.10 |
| Dark | Pt mesh | −2 | 30 | −1164 |
| Dark | Pt mesh | +2 | 30 | 303.2 |
| Dark | Pt mesh | −1 | 30 | −208.1 |
| Dark | Pt mesh | +1 | 30 | 70.29 |
| Dark | Pt mesh | −2 | 60 | −2144 |
| Dark | Pt mesh | +2 | 60 | 535.7 |
| Dark | Pt mesh | −1 | 60 | −337.8 |
| Dark | Pt mesh | +1 | 60 | 89.26 |
| Dark | Pt mesh | −2 | 120 | −3995 |
| Dark | Pt mesh | +2 | 120 | 966.6 |
| Dark | Pt mesh | −1 | 120 | −554.9 |
| Dark | Pt mesh | +1 | 120 | 112.2 |
| Medium | Graphite rod | −2 | 5 | −895.3 |
| Medium | Graphite rod | +2 | 5 | 458.6 |
| Medium | Graphite rod | −1 | 5 | −485.1 |
| Medium | Graphite rod | +1 | 5 | 97.02 |
| Medium | Graphite rod | −2 | 15 | −1995 |
| Medium | Graphite rod | +2 | 15 | 988.0 |
| Medium | Graphite rod | −1 | 15 | −1085 |
| Medium | Graphite rod | +1 | 15 | 181.6 |
| Medium | Graphite rod | −2 | 30 | −2972 |
| Medium | Graphite rod | +2 | 30 | 1579 |
| Medium | Graphite rod | −1 | 30 | −1418 |
| Medium | Graphite rod | +1 | 30 | 267.6 |
| Medium | Graphite rod | −2 | 60 | −4406 |
| Medium | Graphite rod | +2 | 60 | 2562 |
| Medium | Graphite rod | −1 | 60 | −1699 |
| Medium | Graphite rod | +1 | 60 | 396.6 |
| Medium | Graphite rod | −2 | 120 | −6879 |
| Medium | Graphite rod | +2 | 120 | 4250 |
| Medium | Graphite rod | −1 | 120 | −200 |
| Medium | Graphite rod | +1 | 120 | 586.2 |
| Light | Graphite rod | −2 | 0.1 | −5.073 |
| Light | Graphite rod | +2 | 0.1 | 4.888 |
| Light | Graphite rod | −1 | 0.1 | −4.012 |
| Light | Graphite rod | +1 | 0.1 | 1.759 |
| Light | Graphite rod | −2 | 5 | −490.0 |
| Light | Graphite rod | +2 | 5 | 302.6 |
| Light | Graphite rod | −1 | 5 | −298.1 |
| Light | Graphite rod | +1 | 5 | 85.95 |
| Light | Graphite rod | −2 | 15 | −1238 |
| Light | Graphite rod | +2 | 15 | 675.3 |
| Light | Graphite rod | −1 | 15 | −648.8 |
| Light | Graphite rod | +1 | 15 | 165.2 |
| Light | Graphite rod | −2 | 30 | −1923 |
| Light | Graphite rod | +2 | 30 | 1086 |
| Light | Graphite rod | −1 | 30 | −888.6 |
| Light | Graphite rod | +1 | 30 | 242.5 |
| Light | Graphite rod | −2 | 60 | −2795 |
| Light | Graphite rod | +2 | 60 | 1736 |
| Light | Graphite rod | −1 | 60 | −1092 |
| Light | Graphite rod | +1 | 60 | 353.7 |
| Light | Graphite rod | −2 | 120 | −4119 |
| Light | Graphite rod | +2 | 120 | 2842 |
| Light | Graphite rod | −1 | 120 | −1294 |
| Light | Graphite rod | +1 | 120 | 512.4 |

In view of the many possible embodiments to which the principles of the present disclosure may be applied, it should be recognized that the illustrated embodiments are only preferred examples of the disclosure and should not be taken as limiting the scope of the disclosure. Rather, the scope is defined by the following claims. We therefore claim as our invention all that comes within the scope and spirit of these claims.

We claim:

1. A system for electrochemically treating a consumable liquid comprising at least one redox-active sensory compound, the system comprising:
a working electrode comprising an electroactive material configured to react electrochemically with the at least one redox-active sensory compound upon exposure of the working electrode to the liquid;
a counter electrode; and
a power supply configured to apply a potential between the working electrode and the counter electrode, wherein the potential is predetermined to selectively modify the at least one redox-active sensory compound and thereby change at least one sensory property of the liquid;
wherein the working electrode and the counter electrode are positioned to contact the consumable liquid upon exposure of the system to the consumable liquid.

2. The system of claim 1, wherein the electroactive material comprises a carbon material, Pt, Au, Pd, Ag, IrO₂, stainless steel, or a combination thereof.

3. The system of claim 1, wherein the electroactive material comprises glassy carbon, graphite, porous vitreous carbon, diamond, or boron-doped diamond.

4. The system of claim 1, wherein the working electrode comprises an associative catalyst on which the at least one redox-active sensory compound becomes adsorbed upon exposure of the system to the consumable liquid.

5. The system of claim 1, wherein the working electrode comprises a non-associative catalyst with which the at least one redox-active sensory compound reacts upon exposure of the system to the consumable liquid.

6. The system of claim 1, wherein the working electrode comprises a plurality of nanoparticles.

7. The system of claim 1, wherein the at least one redox-active sensory compound is a compound that imparts flavor, mouthfeel, texture, viscosity, aroma, or a combination thereof, to the consumable liquid.

8. The system of claim 1, further comprising a reference electrode, and wherein the potential is predetermined relative to the reference electrode.

9. The system of claim 8, wherein the reference electrode is an Ag/AgCl reference electrode.

10. The system of claim 1, wherein the counter electrode comprises a different electroactive material than the working electrode.

11. The system of claim 10, wherein the electroactive material of the working electrode is a carbon material, Pt, Au, Pd, Ag, stainless steel, or a combination thereof, and wherein the electroactive material of the counter electrode is stainless steel, Pt, Au, Pd, IrO₂, or a combination thereof.

12. The system of claim 10, wherein the counter electrode is substantially inert towards the at least one redox-active sensory compound.

13. The system of claim 1, wherein the counter electrode has a different size or surface area than the working electrode.

14. A method for electrochemically treating a consumable liquid comprising at least one redox-active sensory compound, the method comprising:
contacting an electrode system with the consumable liquid, the electrode system comprising (i) a working electrode comprising an electroactive material configured to react electrochemically with the at least one redox-active sensory compound, and (ii) a counter electrode; and
applying a potential between the working electrode and the counter electrode, wherein the potential is predetermined to selectively modify the at least one redox-active sensory compound and thereby change at least one sensory property of the consumable liquid.

15. The method of claim 14, wherein the potential is commensurate with a redox potential of the redox-active sensory compound.

16. The method of claim 14, wherein the potential ranges from +10 V to −10 V relative to an Ag/AgCl reference electrode.

17. The method of claim 14, wherein applying the potential comprises passing a charge ranging from 1 nC/mL to 1 C/mL into the consumable liquid.

18. The method of claim 14, wherein the power supply is configured to apply a current ranging from greater than 0 nA/mL to 10 A/mL between the working electrode and the counter electrode.

19. The method of claim 14, wherein modifying the at least one redox-active sensory compound comprises electrochemically inducing a structural change or a compositional change in the at least one redox-active sensory compound.

20. The method of claim 19, wherein electrochemically inducing the structural change or the compositional change comprises reducing, oxidizing, hydrolyzing, or decarboxylating the at least one redox-active sensory compound, or any combination thereof.

21. The method of claim 14, wherein the at least one redox-active sensory compound is an organic compound.

22. The method of claim 14, wherein the at least one redox-active sensory compound is a phenol compound, a sugar adduct, a 3-deoxy-D-arabino-heptulosonic acid 7-phosphate (DAHP)-condensate, an alkaloid, an organic acid, or a combination thereof.

23. The method of claim 14, wherein the at least one redox-active sensory compound is caffeine, a chlorogenic acid, or a combination thereof.

24. The method of claim 23, wherein the chlorogenic acid is 3-feruloylquinic acid, 4-feruloylquinic acid, 3-caffeoylquinic acid, 4-caffeoylquinic acid, 5-caffeoylquinic acid, 3,4-dicaffeoylquinic acid, 3,5-dicaffeoylquinic acid, or 4,5-dicaffeoylquinic acid.

25. A consumable liquid produced by the method of claim 14.

26. A system for electrochemically treating a consumable liquid comprising at least one redox-active sensory compound, the system comprising:
a working electrode comprising a first electroactive material configured to react electrochemically with the at least one redox-active sensory compound upon exposure of the working electrode to the liquid;
a reference electrode;
a counter electrode that comprises a second electroactive material that is different from the first electroactive material; and
a power supply configured to apply a potential between the working electrode and the counter electrode, wherein the potential is predetermined relative to the reference electrode to selectively modify the at least one redox-active sensory compound and thereby change at least one sensory property of the consumable liquid;
wherein (i) the counter electrode is substantially inert towards the at least one redox-active sensory compound, and (ii) the working electrode, the reference electrode, and the counter electrode are positioned to contact the consumable liquid upon exposure of the system to the consumable liquid.
